(19) United States
(12) Patent Application Publication
(10) Pub. No.: US 2019/0292561 A1
(43) Pub. Date: Sep. 26, 2019

(54) SCALABLE METHODS FOR PRODUCING RECOMBINANT ADENO-ASSOCIATED VIRAL (AAV) VECTOR IN SERUM-FREE SUSPENSION CELL CULTURE SYSTEM SUITABLE FOR CLINICAL USE

(71) Applicant: SPARK THERAPEUTICS, INC., Philadelphia, PA (US)

(72) Inventors: Guang QU, Sicklerville, NJ (US); Lin LU, Wayne, PA (US); John Fraser WRIGHT, Princeton, NJ (US)

(73) Assignee: SPARK THERAPEUTICS, INC., Philadelphia, PA (US)

(21) Appl. No.: 15/780,542

(22) PCT Filed: Dec. 1, 2016
(86) PCT No.: PCT/US2016/064414

§ 371 Date: May 31, 2018

Related U.S. Application Data
(60) Provisional application No. 62/261,815, filed on Dec. 1, 2015.

(51) Int. Cl. C12N 15/86; C12N 5/00; C12N 5/073; C12N 5/071

(52) U.S. Cl. CPC C12N 15/86; C12N 5/0018; C12N 2750/14141; C12N 5/0686; C12N 2750/14151; C12N 5/0603

(57) ABSTRACT

Methods and compositions for transfecting cells with plasmids are disclosed. In certain embodiments, methods and compositions are disclosed in which transfection efficiency is significantly increased by contacting the cells being transduced with polyethyleneimine (PEI) that is free of nucleic acid during the transfection process. Therapeutically useful adeno-associated viral vectors generated according to the disclosed methods and compositions are also disclosed.

### RELATED APPLICATIONS

This patent application is the National Phase of International Application No. PCT/US2016/064414, filed Dec. 1, 2016, which designated the U.S. and that International Application was published under PCT Article 21(2) in English, which claims the benefit of priority to U.S. patent application No. 62/261,815, filed Dec. 1, 2015, all of which applications are expressly incorporated herein by reference in their entirety.

### SEQUENCE LISTING

The instant application contains a Sequence Listing which has been submitted electronically in ASCII format and is hereby incorporated by reference in its entirety. Said ASCII copy, created on May 31, 2018, is named “Spark0459484_ST25.txt” and is 1.14 KB in size.

### FIELD OF THE INVENTION

This invention relates to the fields of cell transduction (transfection) with nucleic acid, e.g., plasmids. More particularly, the invention provides compositions and methods for producing transduced cells, said cells optionally producing Adeno-Associated Viral (AAV) Vector.

### INTRODUCTION

Several publications and patent documents are cited throughout the specification in order to describe the state of the art to which this invention pertains. Each of these citations is incorporated herein by reference as though set forth in full.

### SUMMARY

The invention provides compositions of nucleic acids (plasmids), such as a nucleic acid that encodes a protein or is transcribed into a transcript of interest, and polyethylenimine (PEI), optionally in combination with cells. In one embodiment, a composition includes a plasmid/PEI mixture, which has a plurality of components: (a) one or more plasmids comprising nucleic acids encoding AAV packaging proteins and/or nucleic acids encoding helper proteins; (b) a plasmid comprising a nucleic acid that encodes a protein or is transcribed into a transcript of interest; and (c) a polyethylenimine (PEI) solution. In particular aspects, the plasmids are in a molar ratio range of about 1:0.01 to about 1:100, or are in a molar ratio range of about 100:1 to about 1:0.01, and the mixture of components (a), (b) and (c) has optionally been incubated for a period of time from about 10 seconds to about 4 hours.

In further embodiments, compositions of nucleic acids (plasmids) and polyethylenimine (PEI) further comprise cells. In particular aspects, the cells are in contact with the plasmid/PEI mixture of components (a), (b) and/or (c).

In additional embodiments, compositions of nucleic acids (plasmids) and polyethylenimine (PEI), optionally in combination with cells, further comprise Free PEI. In particular aspects, the cells are in contact with the Free PEI.

In various further embodiments, the cells have been in contact with the mixture of components (a), (b) and/or (c) for at least about 4 hours, or about 4 hours to about 140 hours, or for about 4 hours to about 96 hours. In particular aspects, the cells have been in contact with the mixture of components (a), (b) and/or (c) and optionally Free PEI, for at least about 4 hours.

Compositions of the invention can be present in a container. In particular, aspects, a container is a flask, plate, bag, or bioreactor, and is optionally sterile, and/or the container is optionally suitable for maintaining cell viability or growth.

Plasmids of invention compositions and methods include, inter alia, nucleic acids that encode viral proteins, such as AAV capsid proteins. Such plasmids and cells may be in contact with Free PEI. In particular aspects, the plasmids and/or cells have been in contact with the Free PEI for at least about 4 hours, or or about 4 hours to about 140 hours, or for about 4 hours to about 96 hours.

Also provided are methods for producing transfected cells, which include providing a plasmid; providing a solution comprising polyethylenimine (PEI); and mixing the nucleic acid (plasmid) with the PEI solution to produce a plasmid/PEI mixture. In particular aspects such mixtures are incubated for a period in the range of about 10 seconds to about 4 hours. In such methods, cells are then contacted with the plasmid/PEI mixture to produce a plasmid/PEI cell culture; then Free PEI is added to the nucleic acid/PEI cell culture produced) to produce a Free PEI/plasmid/PEI cell culture; and then the Free PEI/plasmid/PEI cell culture produced is incubated for at least about 4 hours, thereby producing transfected cells. In particular aspects, the plasmid comprises a nucleic acid that encodes a protein or is transcribed into a transcript of interest.

Further provided are methods for producing transfected cells that produce recombinant AAV vector, which include providing one or more plasmids comprising nucleic acids encoding AAV packaging proteins and/or nucleic acids encoding helper proteins; providing a plasmid comprising a nucleic acid that encodes a protein or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period in the range of about 10 seconds to about 4 hours); contacting cells with the plasmid/PEI mixture), to produce a plasmid/PEI cell culture; adding Free PEI to the plasmid/PEI cell culture produced to produce a Free PEI/plasmid/PEI cell culture; and incubating the Free PEI/plasmid/PEI cell culture for at least about 4 hours, thereby producing transfected cells that produce recombinant AAV vector comprising a nucleic acid that encodes a protein or is transcribed into a transcript of interest.

Additionally provided are methods for producing recombinant AAV vector comprising a nucleic acid that encodes a protein or is transcribed into a transcript of interest, which includes providing one or more plasmids comprising nucleic acids encoding AAV packaging proteins and/or nucleic acids encoding helper proteins; providing a plasmid comprising a nucleic acid that encodes a protein or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period of time in the range of about 10 seconds to about 4 hours); contacting cells with the plasmid/PEI mixture produced as described to produce a plasmid/PEI cell culture; adding Free PEI to the plasmid/PEI cell culture produced as described to produce a Free PEI/plasmid/PEI cell culture; incubating the plasmid/PEI cell culture or the Free PEI/plasmid/PEI cell culture produced for at least about 4 hours to produce transfected cells; harvesting the transfected cells produced and/or culture medium from the transfected cells produced to produce a cell and/or culture medium harvest; and isolating and/or purifying recombinant AAV vector from the cell and/or culture medium harvest produced thereby producing recombinant AAV vector comprising a nucleic acid that encodes a protein or is transcribed into a transcript of interest.

Still further provided are methods for producing transfected cells that produce recombinant AAV vector with a nucleic acid that encodes a protein or is transcribed into a transcript of interest. In one embodiment, a method includes providing a mixture of components (i), one or more plasmids comprising nucleic acids encoding AAV packaging proteins and/or nucleic acids encoding helper proteins, (ii) a plasmid comprising a nucleic acid that encodes a protein or is transcribed into a transcript of interest; and (iii) a polyethylenimine (PEI) solution; mixing the plasmids (i) and (ii) with the PEI solution (iii) so that the plasmids are in a molar ratio range of about 1:0.01 to about 1:100, or in a molar ratio range of about 100:1 to about 1:0.01, to produce a plasmid/PEI mixture (and optionally incubating the plasmid/PEI mixture for a period of time in the range of about 10 seconds to about 4 hours); contacting cells with the plasmid/PEI mixture produced to produce a plasmid/PEI cell culture; adding Free PEI to the plasmid/PEI cell culture to produce a Free PEI/plasmid/PEI cell culture; and incubating the plasmid/PEI cell culture or the Free PEI/plasmid/PEI cell culture for at least about 4 hours to produce transfected cells that produce recombinant AAV vector comprising a nucleic acid that encodes a protein or is transcribed into a transcript of interest.

Methods and compositions of the invention can include one or more steps or features. An exemplary step or feature includes, but is not limited to, a step of harvesting the transfected cells produced and/or harvesting the culture medium from the transfected cells produced to produce a cell and/or culture medium harvest. An additional exemplary step or feature includes, but is not limited to isolating and/or purifying recombinant AAV vector from the cell and/or culture medium harvest thereby producing recombinant AAV vector comprising a nucleic acid that encodes a protein or is transcribed into a transcript of interest.

Still moreover provided are methods for producing recombinant AAV vector that includes a nucleic acid that encodes a protein or is transcribed into a transcript of interest. In one embodiment, a method includes providing a mixture of components (i) one or more plasmids comprising nucleic acids encoding AAV packaging proteins and/or nucleic acids encoding helper proteins, (ii) a plasmid comprising a nucleic acid that encodes a protein or is transcribed into a transcript of interest; and (iii) a polyethylenimine (PEI) solution, mixing the plasmids (i) and (ii) with the PEI solution (iii) so that the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period of time from about 10 seconds to about 4 hours); contacting cells with the plasmid/PEI mixture produced in to produce a plasmid/PEI cell culture; adding Free PEI to the plasmid/PEI cell culture produced to produce a Free PEI/plasmid/PEI cell culture; incubating the plasmid/PEI cell culture or the Free PEI/plasmid/PEI cell culture for at least about 4 hours to produce transfected cells; harvesting the transfected cells produced and/or culture medium from the transfected cells produced to produce a cell and/or culture medium harvest; and isolating and/or purifying recombinant AAV vector from the cell and/or culture medium harvest produced, thereby producing recombinant AAV vector comprising a nucleic acid that encodes a protein or is transcribed into a transcript of interest.

Still additionally provided are methods for producing recombinant AAV vector that includes a nucleic acid that encodes a protein or is transcribed into a transcript of interest. In one embodiment, a method includes providing a mixture of components (i) one or more plasmids comprising nucleic acids encoding AAV packaging proteins and/or nucleic acids encoding helper proteins; (ii) a plasmid comprising a nucleic acid that encodes a protein or is transcribed into a transcript of interest; and (iii) a polyethylenimine (PEI) solution, wherein the plasmids (i) and (ii) are in a molar ratio range of about 1:0.01 to about 1:100, or are in a molar ratio range of about 100:1 to about 1:0.01, and wherein the mixture of components (i), (ii) and (iii) has optionally been incubated for a period of time from about 10 seconds to about 4 hours; contacting cells with the mixture produced to produce a plasmid/PEI cell culture; adding Free PEI to the plasmid/PEI cell culture produced to produce a Free PEI/plasmid/PEI cell culture; incubating the plasmid/PEI cell culture or the Free PEI/plasmid/PEI cell culture for at least about 4 hours to produce transfected cells; harvesting the transfected cells produced and/or culture medium from the transfected cells produced to produce a cell and/or culture medium harvest; and isolating and/or purifying recombinant AAV vector from the cell and/or culture medium harvest produced, thereby producing recombinant AAV vector comprising a nucleic acid that encodes a protein or is transcribed into a transcript of interest.

Compositions and methods may also include one or more additional steps or features. Such steps or features include but are not limited to: where the plasmid/PEI cell culture, or the Free PEI/plasmid/PEI cell culture, or the nucleic acid/PEI cell culture is incubated for a period of time in the range of about 4 hours to about 140 hours, or incubated for a period of time in the range of about 4 hours to about 96 hours. Such steps or features include but are not limited to: where the plasmid/PEI mixture has a PEI:plasmid weight ratio in the range of about 0.1:1 to about 5:1, or has a PEI:plasmid weight ratio in the range of about 5:1 to about 0.1:1, or wherein the Free PEI/plasmid/PEI cell culture has a PEI:plasmid weight ratio in the range of about 0.1:1 to about 5:1, or has a PEI:plasmid weight ratio in the range of about 5:1 to about 0.1:1. Such steps or features include but are not limited to where the plasmid/PEI mixture has a PEI:plasmid weight ratio in the range of about 1:1 to about 5:1, or has a PEI:plasmid weight ratio in the range of about 5:1 to about 1:1; or wherein the Free PEI/plasmid/PEI cell culture has a PEI:plasmid weight ratio in the range of about 1:1 to about 5:1, or has a PEI:plasmid weight ratio in the range of about 5:1 to about 1:1.

Forms of PEI (Free PEI, total PEI, plasmid/PEI mixture, or cells contacted with plasmid/PEI mixture) applicable in the invention compositions and methods include a hydrolyzed linear polyethylenimine. In particular aspects, PEI (Free PEI, total PEI, plasmid/PEI mixture, or cells contacted with plasmid/PEI mixture) comprises a hydrolyzed linear polyethylenimine with a molecular weight in the range of about 4,000 to about 160,000 and/or in the range of about 2,500 to about 250,000 molecular weight in free base form, or a hydrolyzed linear polyethylenimine with a molecular weight of about 40,000 and/or about 25,000 molecular weight in free base form.

In various embodiments, the molar ratio of nitrogen (N) in Total PEI to phosphate (P) in plasmid is in the range of about 1:1 to about 50:1 (N:P) in the Free PEI/plasmid/PEI cell culture. In other embodiments, the molar ratio of nitrogen (N) in Total PEI to phosphate (P) in plasmid is about 5:1, 6:1, 7:1, 8:1, 9:1, or 10:1 (N:P) in the Free PEI/plasmid/PEI cell culture.

Compositions and methods according to the invention can have plasmid/PEI mixtures incubated for a period of time. In particular aspects, incubation is in the range of about 30 seconds to about 4 hours. In more particular aspects, incubation of the plasmid/PEI mixture is in the range of about 1 minute to about 30 minutes.

Compositions and methods according to the invention can have PEI in various percent amounts, either by molar ratio or by weight (mass). In particular embodiments, the amount of Free PEI is in the range of about 10% to about 90% of Total PEI, or the amount of Free PEI is in the range of about 25% to about 75% of Total PEI, or the amount of Free PEI is about 50% of Total PEI.

Compositions and methods according to the invention can have PEI added to plasmids and/or cells at various time points. In particular embodiments, Free PEI is added to the cells before, at the same time as, or after the plasmid/PEI mixture is contacted with the cells.

Compositions and methods according to the invention include mammalian cells (e.g., HEK 293E or HEK 293F cells). Such cells can be adherent or be in suspension culture. In particular aspects, cells are grown or maintained in a serum-free culture medium.

Compositions and methods according to the invention can have cells at particular densities and/or cell growth phases and/or viability. In particular embodiments, cells are at a density in the range of about 1×10⁵ cells/mL to about 1×10⁸ cells/mL when contacted with the plasmid/PEI mixture and/or when contacted with the Free PEI. In additional particular embodiments, viability of the cells when contacted with the plasmid/PEI mixture or with the Free PEI is about 60% or greater than 60%, or wherein the cells are in log phase growth when contacted with the plasmid/PEI mixture, or viability of the cells when contacted with the plasmid/PEI mixture or with the Free PEI is about 90% or greater than 90%, or wherein the cells are in log phase growth when contacted with the plasmid/PEI mixture or with the Free PEI.

Encoded AAV packaging proteins include, for example, AAV rep and/or AAV cap. Such AAV packaging proteins include, for example, AAV rep and/or AAV cap proteins of any AAV serotype.

Encoded helper proteins include, for example, adenovirus E2 and/or E4, VARNA proteins, and/or non-AAV helper proteins.

Compositions and methods according to the invention can have nucleic acid (plasmids) at particular amounts or ratios. In particular embodiments, the total amount of plasmid comprising the nucleic acid that encodes a protein or is transcribed into a transcript of interest and the one or more plasmids comprising nucleic acids encoding AAV packaging proteins and/or nucleic acids encoding helper proteins is in the range of about 0.1 μg to about 15 μg per mL of cells. In additional particular embodiments, the molar ratio of the plasmid comprising the nucleic acid that encodes a protein or is transcribed into a transcript of interest to the one or more plasmids comprising nucleic acids encoding AAV packaging proteins and/or nucleic acids encoding helper proteins is in the range of about 1:5 to about 1:1, or is in the range of about 1:1 to about 5:1.

Plasmids can include nucleic acids on different or the same plasmids. In one embodiment, a first plasmid comprises the nucleic acids encoding AAV packaging proteins and a second plasmid comprises the nucleic acids encoding helper proteins. In more particular embodiments, the molar ratio of the plasmid comprising the nucleic acid that encodes a protein or is transcribed into a transcript of interest to a first plasmid comprising the nucleic acids encoding AAV packaging proteins to a second plasmid comprising the nucleic acids encoding helper proteins is in the range of about 1-5:1:1, or 1:1-5:1, or 1:1:1-5.

Compositions and methods according to the invention include AAV vectors of any serotype, or a variant thereof. In one embodiment, a recombinant AAV vector comprises any of AAV serotypes 1-12, an AAV VP1, VP2 and/or VP3 capsid protein, or a modified or variant AAV VP1, VP2 and/or VP3 capsid protein, or wild-type AAV VP1, VP2 and/or VP3 capsid protein. In additional particular embodiments, an AAV vector comprises an AAV serotype or an AAV pseudotype, where the AAV pseudotype comprises an AAV capsid serotype different from an ITR serotype.

Compositions and methods according to the invention that provide or include AAV vectors can also include other elements. Examples of such elements include but are not limited to: an intron, an expression control element, one or more adeno-associated virus (AAV) inverted terminal repeats (ITRs) and/or a filler polynucleotide sequence. Such elements can be within or flank the nucleic acid that encodes a protein or is transcribed into a transcript of interest, or the expression control element can be operably linked to nucleic acid that encodes a protein or is transcribed into a transcript of interest, or the AAV ITR(s) can flank the 5′ or 3′ terminus of nucleic acid that encodes a protein or is transcribed into a transcript of interest, or the filler polynucleotide sequence can flank the 5′ or 3′terminus of nucleic acid that encodes a protein or is transcribed into a transcript of interest.

Expression control elements include constitutive or regulatable control elements, such as a tissue-specific expression control element or promoter (e.g. that provides for expression in liver).

ITRs can be any of: AAV2 or AAV6 serotypes, or a combination thereof. AAV vectors can include any VP1, VP2 and/or VP3 capsid protein having 75% or more sequence identity to any of AAV1, AAV2, AAV3, AAV4, AAV5, AAV6, AAV10, AAV11, or AAV-2i8 VP1, VP2 and/or VP3 capsid proteins, or comprises a modified or variant VP1, VP2 and/or VP3 capsid protein selected from any of: AAV1, AAV2, AAV3, AAV4, AAV5, AAV6, AAV10, AAV11, and AAV-2i8 AAV serotypes.

In compositions and methods of the invention, cells can be sub-cultured, such as cell density reduced by dilution or removal of cells from the culture. In one embodiment, cells are subcultured to a reduced cell density prior to contact with the plasmid/PEI mixture.

In compositions and methods of the invention, cells can be employed at various densities. In one embodiment, cells are cultured or are subcultured to a cell density in the range of about 0.1×10⁶ cells/ml to about 5.0×10⁶ cells/ml prior to contact with the plasmid/PEI mixture.

In compositions and methods of the invention, cells can be contacted with PEI (Free PEI, total PEI, plasmid/PEI mixture) for a period of time, short or long term. In one embodiment, cells are contacted with the plasmid/PEI mixture between a period of 2 days to 5 days after subculture. In another embodiment, cells are contacted with the plasmid/PEI mixture between a period of 3 days to 4 days after subculture.

Compositions and methods of the invention provide enhanced cell transfection efficiency and/or recombinant production of vectors by cells. In one embodiment, the amount of plasmid introduced into transfected cells is at least 50% greater with the step of adding Free PEI to the plasmid/PEI cell culture compared to without adding Free PEI to the plasmid/PEI cell culture. In another embodiment, the amount of recombinant AAV vector produced is at least 50% or greater with the step of adding Free PEI to the plasmid/PEI cell culture compared to without adding Free PEI to the plasmid/PEI cell culture. In a further embodiment, the amount of recombinant AAV vector produced is 1-5, 5-10 or 10-20 fold greater with the step of adding Free PEI to the plasmid/PEI cell culture compared to without adding Free PEI to the plasmid/PEI cell culture.

### DESCRIPTION OF DRAWINGS

FIG. 1 shows transfection efficiency of PEI “Max” 40 KDa (A) and PEI 25 KDa (B) dissolved in either TrisHCl or H₂O when using plasmid DNA at 2.8, 5.6 and 11.2 μg/mL. PEI “Max” 40 KDa showed consistent higher transfection efficiency compared with PEI 25 KDa.

FIG. 2A-2B shows effect of Free PEI on transfection efficiency and rAAV vector production of in 12-well plates. A) Transfection of 293F cells with three plasmids (pAAV-eGFP-WRPE, pAAV-Rep2/Cap2, pAD2-Helper) in serum-free suspension culture in 12-well plates. B) Effect of Free PEI on rAAV titer. PEI/DNA weight ratio was 2:1 or 4:1 with or without adding Free PEI at transfection. DNA amount of 2.8 μg/mL was used with molar ratio 1:1:1 for three plasmids. Diluted PEI was first mixed with diluted DNA at weight ratio 1:1 to form the complexes. The excess PEI was diluted in 50 μL culture medium and then added directly to the cells.

FIG. 3A-3B shows effect of Free PEI on transfection efficiency and rAAV titer in spinner flasks. A) Transfection efficiency was increased by using Free PEI. B) Effect of Free PEI on rAAV titer. PEI/DNA weight ratio was 2:1 and DNA amount was 2.8 μg/mL. ½ and ⅓ of PEI amount was used as Free PEI at transfection.

FIG. 4 shows 293F cell growth curve and viability in bioreactor. Cells were seeded at 0.25×10⁶ cells/mL (Unit 1), 0.35×10⁶ cells/mL (Unit 2) and 0.5×10⁶ cells/mL (Unit 3). Cell density (N) and viability (V) were recorded every 12 hours during 7 days of cell culture in bioreactor.

FIG. 5A-5B shows cell transfection in Bioreactor. A) 293F cells transfected with three plasmids for up to 72 h. GFP positive cells were detected with inverted fluorescence microscope. B) The transfection efficiency was measured with flow cytometry. 50%-60% of GFP positive cells was detected at 48 h-72 h post-transfection. The transfection efficiency was similar between day 3 transfection and day 4 transfection.

FIG. 6A-6B shows rAAV titer and vector function assay. A) Vector titer was significantly higher on day 4 transfection then day 3 assessed by qPCR. The highest titer was 1.38E+11 vg/mL in the condition of transfection at day 4 and agitation speed at 150 rpm. B) Vector function was measured by transduction assay. The same volume of cell lysates was added to HEK 293 cells. GFP positive cells were detected with inverted fluorescence microscope. The transduction results were consistent with the rAAV titer that is higher titer correlated with higher transduction rate.

### DETAILED DESCRIPTION

Disclosed herein are compositions and methods of transducing cells with a molecule, such as a nucleic acid (e.g., plasmid), at high efficiency. Such high efficiency transduced cells can, when transduced with a nucleic acid that encodes a protein or comprises a sequence that is transcribed into a transcript of interest, can produce protein and/or transcript at high efficiency. Additionally, such cells when transduced with sequences, such as plasmids that encode viral packaging proteins and/or helper proteins can produce recombinant vectors that include the nucleic acid that encodes a protein or comprises a sequence that is transcribed into a transcript of interest, which in turn produces recombinant viral vectors at high yield.

The invention provides a cell transduction and/or a viral (e.g., AAV) vector production platform that includes features that distinguish it from current ‘industry-standard’ viral (e.g., AAV) vector production processes. The compositions and methods of the invention are characterized by mixing PEI with nucleic acids under certain conditions. Mixing PEI with nucleic acids results in PEI-induced efficient compaction of nucleic acids to form stable complexes termed polyplexes. The method of introducing nucleic acids into cells comprises providing nucleic acids mixed with PEI under certain conditions, and applying the resulting mixture to cells. Further, the compositions and methods of the invention are characterized by cells contacted with Free PEI, or contacting cells with Free PEI, in a particular sequence with respect to the step of applying the PEI/nucleic acids mixture to cells. The compositions and methods of the invention are characterized by: 1) high efficiency nucleic acid cell transduction/transfection; 3) a unique combination of reagents and process steps that confers unexpected substantial yield of vector; and 4) a modular platform that can be used for production of different AAV serotypes/capsid variants.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids and polynucleotides include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids and polynucleotides include naturally occurring, synthetic, and intentionally modified or altered sequences (e.g., variant nucleic acid).

A nucleic acid or plasmid can also refer to a sequence which encodes a protein. Such proteins can be wild-type or a variant, modified or chimeric protein. A “variant protein” can mean a modified protein such that the modified protein has an amino acid alteration compared to wild-type protein.

Proteins encoded by a nucleic acid or plasmid include therapeutic proteins. Non-limiting examples include a blood clotting factor (e.g., Factor XIII, Factor IX, Factor X, Factor VIII, Factor VIIa, or protein C), CFTR (cystic fibrosis transmembrane regulator protein), an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor (e.g., insulin-like growth factors 1 and 2, platelet derived growth factor, epidermal growth factor, nerve growth factor, neurotrophic factor −3 and −4, brain-derived neurotrophic factor, glial derived growth factor, transforming growth factor α and β, etc.), a cytokine (e.g., α-interferon, β-interferon, interferon-γ, interleukin-2, interleukin-4, interleukin 12, granulocyte-macrophage colony stimulating factor, lymphotoxin, etc.), a suicide gene product (e.g., herpes simplex virus thymidine kinase, cytosine deaminase, diphtheria toxin, cytochrome P450, deoxycytidine kinase, tumor necrosis factor, etc.), a drug resistance protein (e.g, that provides resistance to a drug used in cancer therapy), a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitopes, or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, gene deficiencies causative of lysosomal storage diseases (e.g., sulfatases, N-acetylglucosamine-1-phosphate transferase, cathepsin A, GM2-AP, NPC1, VPC2, Sphingolipid activator proteins, etc.), one or more zinc finger nucleases for genome editing, or donor sequences used as repair templates for genome editing.

A nucleic acid or plasmid can also refer to a sequence which produces a transcript when transcribed. Such transcripts can be RNA, such as inhibitory RNA (RNAi, e.g., small or short hairpin (sh)RNA, microRNA (miRNA), small or short interfering (si)RNA, trans-splicing RNA, or antisense RNA).

Non-limiting examples include inhibitory nucleic acids that inhibit expression of: huntingtin (HTT) gene, a gene associated with dentatorubropallidolusyan atropy (e.g., atrophin 1, ATN1); androgen receptor on the X chromosome in spinobulbar muscular atrophy, human Ataxin-1, -2, -3, and -7, Cav2.1 P/Q voltage-dependent calcium channel is encoded by the (CACNA1A), TATA-binding protein, Ataxin 8 opposite strand, also known as ATXN8OS, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DAN damage-inducible transcript 4 protein, in diabetic macular oedma (DME) or age-related macular degeneration; vascular endothelial growth factor receptor I (VEGFR1) in age-related macular degeneration or choroidal neivascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; mutant rhodopsin gene (RHO) in autosomal dominantly inherited retinitis pigmentosa (adRP); or the inhibitory nucleic acid binds to a transcript of any of the foregoing genes or sequences.

Nucleic acids (plasmids) can be single, double, or triplex, linear or circular, and can be of any length. In discussing nucleic acids (plasmids), a sequence or structure of a particular polynucleotide may be described herein according to the convention of providing the sequence in the 5′ to 3′ direction.

A “plasmid” is a form of nucleic acid or polynucleotide that typically has additional elements for expression (e.g., transcription, replication, etc.) or propagation (replication) of the plasmid. A plasmid as used herein also can be used to reference such nucleic acid or polynucleotide sequences. Accordingly, in all aspects the invention compositions and methods are applicable to nucleic acids and polynucleotides, e.g., for introducing nucleic acid or polynucleotide into cells, for transducing (transfecting) cells with nucleic acid or polynucleotide, for producing transduced (transfected) cells that have a nucleic acid or polynucleotide, to produce cells that produce viral (e.g., AAV) vectors, to produce viral (e.g., AAV) vectors, to produce cell culture medium that has viral (e.g., AAV) vectors, etc.

Compositions and methods of the invention include polyethyleneimine (PEI). PEI is a cationic polymer and is able to form a stable complex with nucleic acid, referred to as a polyplex. Although not wishing to be bound by any theory, the polyplex is believed to be introduced into cells through endocytosis.

PEI can be linear PEI or branched PEI. PEI can be in a salt form or free base. In particular embodiments, PEI is linear PEI, such as an optionally hydrolyzed linear PEI. The hydrolyzed PEI may be fully or partially hydrolyzed. Hydrolyzed linear PEI has a greater proportion of free (protonatable) nitrogens compared to non-hydrolyzed linear PEI, typically having at least 1-5% more free (protonatable) nitrogens compared to non-hydrolyzed linear PEI, more typically having 5-10% more free (protonatable) nitrogens compared to non-hydrolyzed linear PEI, or most typically having 10-15% more free (protonatable) nitrogens compared to non-hydrolyzed linear PEI.

In particular embodiments, PEI can have a molecular weight in the range of about 4,000 to about 160,000 and/or in the range of about 2,500 to about 250,000 molecular weight in free base form. In further particular embodiments, PEI can have a molecular weight of about 40,000 and/or about 25,000 molecular weight in free base form. Specifically, linear PEI with a molecular weight of about 40,000 and/or about 25,000 molecular weight in free base form. In addition, chemically modified linear PEI or branched PEI can be also used. PEI is commercially available (e.g., Polysciences, Inc., Warrington, Pa., USA).

In invention compositions and methods, a nucleic acid, such as a plasmid is mixed with PEI to form a PEI mixture or solution. Such a mixture or solution can be referred to as “a plasmid/PEI mixture,” or a “a nucleic acid/PEI mixture.” The terms “plasmid/PEI mixture” and “nucleic acid/PEI mixture” therefore mean that the PEI has been mixed with the nucleic acid/plasmid. The PEI as set forth herein may therefore be mixed with nucleic acid (plasmid), prior to or substantially simultaneously with contact of the cells for transduction.

As used herein, the term “Free PEI” means PEI that is substantially or entirely free of nucleic acid (plasmid). The PEI as set forth herein may therefore also be in the form of Free PEI. The “plasmid/PEI mixture” or “nucleic acid/PEI mixture” is therefore distinct from Free PEI. If Free PEI is substantially free, the amount of nucleic acid (plasmid) sequences present, will be no more than about 5% as determined by molecular weight or by mass. Of course, the amount may be less than 5%, e.g., about 4.5% or less, about 4% or less, about 3.5% or less, about 3% or less, about 2.5% or less, about 2% or less, about 1.5% or less, about 1% or less, or about 0.5% or less.

As used herein, the term “Total PEI” means the sum of PEI present in PEI/plasmid mixture and Free PEI. The Total PEI therefore includes PEI that is mixed with the plasmid and PEI that is substantially or entirely free of nucleic acid sequences, such as a plasmid.

The disclosure of PEI quantities, ratios, compositions, solutions, solvents and buffers, pH, salts, and timing and duration of cell contact and incubation applies to any one of, any two of, or all three of: 1) PEI in a plasmid/PEI mixture or in a nucleic acid/PEI mixture; 2) PEI as Free PEI (i.e., PEI that is substantially or entirely free of nucleic acid or polynucleotide sequences, such as a plasmid; and 3) Total PEI (PEI in a plasmid/PEI mixture or in a nucleic acid/PEI mixture+Free PEI).

In particular embodiments, PEI is a solution, such as an aqueous (e.g., water) solutions. In additional particular embodiments, PEI is acidified or neutralized PEI. The term “acidified PEI” means a PEI solution that is prepared by dissolving PEI in an acidic solvent. Acidity of the acidified PEI solution is typically a pH from about 0 to about 3.0, more typically a pH from about 0.5 to about 2.0. The term “neutralized PEI” means a PEI solution that is prepared by dissolving PEI in a neutral solvent or buffer. Neutralized PEI solutions can have a pH in the range of about 6.0 to about 8.0, typically a pH in the range of about 6.5 to about 7.5, more typically a pH in the range of about 6.8 to about 7.2, and most typically a pH in the range of about 7.0 to about 7.2, e.g., about 7.1.

Any solvent or buffer can be used for establishing or maintaining pH of a PEI solution within an aforementioned range without destroying the transfection activity of PEI. Examples of acidic solvents include mineral acids such as Hydrochloric acid (HCl), and organic acids with pH in acidic range such as glycine-hydrochloric acid solution. Non-limiting examples of neutral solvents/buffers include Tris (trizma base) and HEPES. Buffers can range from about 1 mM to about 100 mM, more typically from about 2 mM to about 50 mM, and most typically from about 5 mM to about 20 mM.

PEI solutions can optionally include salts. Non-limiting examples of salts include sodium (Na), potassium (K) and magnesium (Mg) salts. In particular aspects, salt concentrations of a PEI solution ranges from about 50 mM to about 500 mM, more typically from about 100 mM to about 250 mM, and most typically from about 125 mM to about 175 mM.

A mixture of nucleic acids (plasmid) and PEI is carried out by mixing nucleic acids (plasmid) and PEI in a solution. The mixing can occur in any solution compatible with PEI based cell transduction. Non-limiting examples are as set forth herein. After mixing, the nucleic acids (plasmid)/PEI mixture can be incubated for a time period of from about 1 minute to about 8 hours; from about 10 seconds to about 4 hours; from about 1 minute to about 60 minutes; from about 1 minute to about 30 minutes; from about 10 minutes to about 45 minutes; from about 10 minutes to about 30 minutes; and/or from about 20 minutes to about 30 minutes. Typically times include about 1 minute, about 5 minutes, about 10 minutes, about 15 minutes, about 20 minutes and about 30 minutes.

PEI and nucleic acids (plasmid) are mixed at a ratio that is not limited. Typical ratios include a mixture of plasmids in a molar (or weight) ratio range of about 1:0.01 to about 1:100, or in a molar (or weight) ratio range of about 100:1 to about 1:0.01, to produce plasmid/PEI mixture. More typical molar (or weight) ratios include a mixture of plasmids in a molar (or weight) ratio range of about 1:1 to about 1:5, or in a molar (or weight) ratio range of about 1:2 to about 1:4, to produce plasmid/PEI mixture. In additional embodiments, the PEI:plasmid weight ratio is in the range of about 0.1:1 to about 5:1, or in the range of about 5:1 to about 0.1:1. In further embodiments, Free PEI/plasmid/PEI cell culture has a PEI:plasmid weight ratio in the range of about 0.1:1 to about 5:1, or has a PEI:plasmid weight ratio in the range of about 5:1 to about 0.1:1. In particular embodiments, the plasmid/PEI mixture has a PEI:plasmid weight ratio in the range of about 1:1 to about 5:1, or in the range of about 5:1 to about 1:1. In other particular embodiments, the Free PEI/plasmid/PEI cell culture has a PEI:plasmid weight ratio in the range of about 1:1 to about 5:1, or in the range of about 5:1 to about 1:1.

The amount of nucleic acids (plasmid) used to produce compositions and methods of cell transduction varies. In particular embodiments, the molar ratio of nitrogen (N) in Total PEI to phosphate (P) in plasmid is in the range of about 1:1 to about 50:1 (N:P) in the Free PEI/plasmid/PEI cell culture, or the molar ratio of nitrogen (N) in Total PEI to phosphate (P) in plasmid is about 1:1 to 10:1 (N:P) in the Free PEI/plasmid/PEI cell culture, or the molar ratio of nitrogen (N) in Total PEI to phosphate (P) in plasmid is about 5:1, 6:1, 7:1, 8:1, 9:1, or 10:1 (N:P) in the Free PEI/plasmid/PEI cell culture. In additional particular embodiments, the total amount of plasmid comprising the nucleic acid that encodes a protein or is transcribed into a transcript of interest and the one or more plasmids comprising nucleic acids encoding AAV packaging proteins and/or nucleic acids encoding helper proteins is in the range of about 0.1 μg to about 15 μg per mL of cells.

Applying a mixture of nucleic acids (plasmid)/PEI to cells is carried out by adding the nucleic acids (plasmid)/PEI mixture to cells such that the mixture of nucleic acids (plasmid)/PEI contacts the cells. Cells to which the mixture of nucleic acids (plasmid)/PEI solutions is added (contacted) can be adherent cells or cells in suspension. Such cells can include co-cultures with other cells.

Cells are contacted for a time period with a mixture of nucleic acids (plasmid)/PEI that is not limited, to achieve cell transduction. Contact of cells with Free PEI typically occurs concurrently with (or immediately after), or after cells have been contacted with the nucleic acids (plasmid)/PEI mixture. Should there be a time interval between contact of cells with nucleic acids (plasmid)/PEI mixture and contact of the cells with Free PEI, the time interval can be from about 1 second to about 140 hours, typically from about 1 second to about 96 hours, more typically from about 1 second to about 48 or about 72 hours, most typically from about 1 second to about 24 hours, or less, e.g., about 16, about 12, about 8, or about 6 hours, or less.

For long term contact, cells may be affected by cytotoxicity of PEI resulting in an increased amount of dead (non-viable) cells thereby reducing transfection efficiency. The incubation time after cells are contacted with Total PEI can range from seconds to days. Specifically, cells can be contacted with nucleic acids (plasmid)/PEI, or Total PEI, for example, for a time period of from about 1 minute to about 48 hours; from about 1 minute to about 24 hours; from about 1 minute to about 16 hours; from about 1 minute to about 8 hours; from about 1 minute to about 4 hours; from about 1 minute to about 120 minutes; from about 5 minutes to about 60 minutes; from about 10 minutes to about 45 minutes; or from about 10 minutes to about 30 minutes.

To reduce cytotoxicity of PEI, culture medium may be replaced with fresh culture medium after contacting the cells with nucleic acids (plasmid)/PEI. Culture medium replacement after transfection can minimize PEI cytotoxicity without significant loss of cell transfection efficiency.

Cells for transfection, either prior to or at the time of contact with plasmid/PEI mixture or contact with Free PEI, have a density in the range of about 1×10⁵ cells/mL to about 1×10⁸ cells/mL when contacted with the plasmid/PEI mixture or when contacted with the Free PEI. Typically, cells have a density in the range of about 2×10⁵ cells/mL to about 5×10⁶ cells/mL. More typically, cells have a density in the range of about 3×10⁵ cells/mL to about 3×10⁶ cells/mL, e.g., about 4×10⁵ cells/mL to about 2×10⁶ cells/mL, or about 3×10⁵ cells/mL to about 1×10⁶ cells/mL.

Cells for transfection, either prior to or at the time of contact with plasmid/PEI mixture and/or contact with Free PEI, can optionally be in log (exponential) phase of growth. Cells for transfection, either prior to or at the time of contact with plasmid/PEI mixture and/or contact with Free PEI, can optionally have 60% or greater than 60% viability, e.g., 70%, 80%, or 90% or greater than 90% viability.

Cells that may be contacted as set forth herein include mammalian cells, such as human cells. Such cells may be primary cells or cell lines that are capable of growth or maintaining viability in vitro, or have been adapted for in vitro tissue culture. Examples of cell lines include HEK (human embryonic kidney) cells, which include HEK293 cells, such as HEK293F (293F) and HEK293T (293T) cells.

More generally, such cells contacted as set forth herein can be referred to as “host cells.” A “host cell” denotes, for example, microorganisms, yeast cells, insect cells, and mammalian cells, that can be, or have been, used as recipients of nucleic acid (plasmid) encoding packaging proteins, such as AAV packaging proteins, a nucleic acid (plasmid) encoding helper proteins, a nucleic acid (plasmid) that encodes a protein or is transcribed into a transcript of interest, or other transfer nucleic acid (plasmid). The term includes the progeny of the original cell, which has been transduced or transfected. Thus, a “host cell” as used herein generally refers to a cell which has been transduced or transfected with an exogenous nucleic acid sequence. It is understood that the progeny of a single parental cell may not necessarily be completely identical in morphology or in genomic or total nucleic acid complement as the original parent, due to natural, accidental, or deliberate mutation.

Numerous cell growth medium appropriate for sustaining cell viability or providing cell growth and/or proliferation are commercially available or can be readily produced. Examples of such medium include serum free eukaryotic growth mediums, such as medium for sustaining viability or providing for the growth of mammalian (e.g., human) cells. Non-limiting examples include Ham's F12 or F12K medium (Sigma-Aldrich), FreeStyle (FS) F17 medium (Thermo-Fisher Scientific), MEM, DMEM, RPMI-1640 (Thermo-Fisher Scientific) and mixtures thereof. Such medium can be supplemented with vitamins and/or trace minerals and/or salts and/or amino acids, such as essential amino acids for mammalian (e.g., human) cells.

The terms “transduce” and “transfect” refer to introduction of a molecule such as a nucleic acid (plasmid) into a host cell. A cell has been “transduced” or “transfected” when exogenous nucleic acid has been introduced inside the cell membrane. Accordingly, a “transduced cell” is a cell into which a “nucleic acid” or “polynucleotide” has been introduced, or a progeny thereof in which an exogenous nucleic acid has been introduced. In particular embodiments, a “transduced” cell (e.g., in a mammal, such as a cell or tissue or organ cell) is a genetic change in a cell following incorporation of an exogenous molecule, for example, a nucleic acid (e.g., a transgene). A “transduced” cell(s) can be propagated and the introduced nucleic acid transcribed and/or protein expressed.

In a “transduced” or “transfected” cell, the nucleic acid (plasmid) may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extrachromosomally, or only transiently. A number of techniques are known (See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties into suitable host cells.

The term “vector” refers to small carrier nucleic acid molecule, a plasmid, virus (e.g., AAV vector), or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, ITR(s), selectable marker (e.g., antibiotic resistance), polyadenylation signal. For purposes of the invention, a “vector” as set forth herein is within the scope of a “plasmid” as this term is used herein.

A viral vector is derived from or based upon one or more nucleic acid elements that comprise a viral genome. Particular viral vectors include lentivirus, pseudo-typed lentivirus and parvo-virus vectors, such as adeno-associated virus (AAV) vectors.

The term “recombinant,” as a modifier of vector, such as recombinant viral, e.g., lenti- or parvo-virus (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome, i.e., is heterologous. Although the term “recombinant” is not always used herein in reference to vectors, such as viral and AAV vectors, as well as sequences such as polynucleotides, recombinant forms including polynucleotides, are expressly included in spite of any such omission.

A recombinant viral “vector” or “AAV vector” is derived from the wild type genome of a virus, such as AAV by using molecular methods to remove the wild type genome from the virus (e.g., AAV), and replacing with a non-native nucleic acid, such as a nucleic acid transcribed into a transcript or that encodes a protein. Typically, for AAV one or both inverted terminal repeat (ITR) sequences of AAV genome are retained in the AAV vector. A “recombinant” viral vector (e.g., AAV) is distinguished from a viral (e.g., AAV) genome, since all or a part of the viral genome has been replaced with a non-native (i.e., heterologous) sequence with respect to the viral (e.g., AAV) genomic nucleic acid. Incorporation of a non-native sequence therefore defines the viral vector (e.g., AAV) as a “recombinant” vector, which in the case of AAV can be referred to as a “rAAV vector.”

A recombinant vector (e.g., lenti-, parvo-, AAV) sequence can be packaged-referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV.” Such particles include proteins that encapsidate or package the vector genome. Particular examples include viral envelope proteins, and in the case of AAV, capsid proteins, such as AAV VP1, VP2 and VP3.

A vector “genome” refers to the portion of the recombinant plasmid sequence that is ultimately packaged or encapsidated to form a viral (e.g., AAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the nucleic acid that is packaged or encapsidated by virus (e.g., AAV).

The terms “empty capsid” and “empty particle,” refer to an AAV virion that includes an AAV protein shell but that lacks in whole or part a nucleic acid that encodes a protein or is transcribed into a transcript of interest flanked by AAV ITRs. Accordingly, the empty capsid does not function to transfer a nucleic acid that encodes a protein or is transcribed into a transcript of interest into the host cell. However, empty capsid formulations have utility in other applications, such as ELISA.

The term “packaging proteins” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

As used herein, “AAV packaging proteins” refer to AAV-derived sequences which function in trans for productive AAV replication. Thus, AAV packaging proteins are encoded by the major AAV open reading frames (ORFs), rep and cap. The rep proteins have been shown to possess many functions, including, among others: recognition, binding and nicking of the AAV origin of DNA replication; DNA helicase activity; and modulation of transcription from AAV (or other heterologous) promoters. The cap (capsid) proteins supply necessary packaging functions. AAV packaging proteins are used herein to complement AAV functions in trans that are missing from AAV vectors.

The “nucleic acids encoding AAV packaging proteins” refer generally to a nucleic acid molecule that includes nucleotide sequences providing AAV functions deleted from an AAV vector which is to be used to produce a transducing recombinant AAV vector. The nucleic acids encoding AAV packaging proteins are commonly used to provide transient expression of AAV rep and/or cap genes to complement missing AAV functions that are necessary for AAV replication; however, the nucleic acid constructs lack AAV ITRs and can neither replicate nor package themselves. Nucleic acids encoding AAV packaging proteins can be in the form of a plasmid, phage, transposon, cosmid, virus, or virion. A number of nucleic acid constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of vectors have been described which encode Rep and/or Cap expression products (e.g., U.S. Pat. Nos. 5,139,941 and 6,376,237).

The term “nucleic acids encoding helper proteins” refers generally to a nucleic acid molecule(s) that includes nucleotide sequences encoding proteins that provide helper function(s). A vector with nucleic acid(s) encoding helper protein(s) can be transfected into a suitable host cell, wherein the vector is then capable of supporting AAV virion production in the host cell. Expressly excluded from the term are infectious viral particles, as they exist in nature, such as adenovirus, herpesvirus or vaccinia virus particles.

Thus, helper protein vectors can be in the form of a plasmid, phage, transposon or cosmid. In particular, it has been demonstrated that the full-complement of adenovirus genes are not required for helper functions. For example, adenovirus mutants incapable of DNA replication and late gene synthesis have been shown to be permissive for AAV replication. Ito et al., (1970) J. Gen. Virol. 9:243; Ishibashi et al, (1971) Virology 45:317.

Mutants within the E2B and E3 regions have been shown to support AAV replication, indicating that the E2B and E3 regions are probably not involved in providing helper function. Carter et al., (1983) Virology 126:505. However, adenoviruses defective in the E1 region, or having a deleted E4 region, are unable to support AAV replication. Thus, for adenoviral helper proteins, EIA and E4 regions are likely required for AAV replication, either directly or indirectly. Laughlin et al., (1982) J. Virol. 41:868; Janik et al., (1981) Proc. Natl. Acad. Sci. USA 78:1925; Carter et al., (1983) Virology 126:505. Other characterized Ad mutants include: EIB (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)).

Studies of the helper proteins provided by adenoviruses having mutations in the E1B have reported that E1 B55k is required for AAV virion production, while E1B 19k is not. In addition, International Publication WO 97/17458 and Matshushita et al., (1998) Gene Therapy 5:938-945, describe helper function vectors encoding various Ad genes. An example of a helper vector comprise an adenovirus VA RNA coding region, an adenovirus E4 ORF6 coding region, an adenovirus E2A 72 kD coding region, an adenovirus E1A coding region, and an adenovirus E1B region lacking an intact E I BS5k coding region (see, e.g., International Publication No. WO 01/83797).

A “transgene” is used herein to conveniently refer to a nucleic acid that is intended or has been introduced into a cell or organism. Transgenes include any nucleic acid, such as a gene that is transcribed into a transcript or that encodes a polypeptide or protein.

An “expression control element” refers to nucleic acid sequence(s) that influence expression of an operably linked nucleic acid. Control elements, including expression control elements as set forth herein such as promoters and enhancers, Vector sequences including AAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of certain vectors, such as AAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed sequence. A promoter typically increases an amount expressed from operably linked nucleic acid as compared to an amount expressed when no promoter exists.

An “enhancer” as used herein can refer to a sequence that is located adjacent to the heterologous polynucleotide Enhancer elements are typically located upstream of a promoter element but also function and can be located downstream of or within a nucleic acid sequence. Hence, an enhancer element can be located 100 base pairs, 200 base pairs, or 300 or more base pairs upstream or downstream of a nucleic acid. Enhancer elements typically increase expressed of an operably linked nucleic acid above expression afforded by a promoter element.

An expression construct may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

The incorporation of tissue specific regulatory elements in the plasmids of the invention provides for at least partial tissue tropism for expression of the nucleic acid. Examples of promoters that are active in liver are the TTR promoter, human alpha 1-antitrypsin (hAAT) promoter; albumin, Miyatake, et al. J. Virol., 71:5124-32 (1997); hepatitis B virus core promoter, Sandig, et al., Gene Ther. 3:1002-9 (1996); alpha-fetoprotein (AFP), Arbuthnot, et al., Hum. Gene. Ther., 7:1503-14 (1996)], among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al, Cell, 41:521-530 (1985)), the SV40 promoter, the dihydrofolate reductase promoter, the cytoplasmic β-actin promoter and the phosphoglycerol kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase, development.

Expression control elements also include the native elements(s) for the nucleic acid. A native control element (e.g., promoter) may be used when it is desired that expression of the heterologous polynucleotide should mimic the native expression. The native element may be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences may also be used.

The term “operably linked” means that the regulatory sequences necessary for expression of a coding sequence are placed in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

An intron can also function as a filler or stuffer polynucleotide sequence in order to achieve a length for AAV vector packaging into a virus particle. Introns and intron fragments that function as a filler or stuffer polynucleotide sequence also can enhance expression.

The “polypeptides,” “proteins” and “peptides” encoded by the “nucleic acid” or “plasmids,” include full-length native sequences, as with naturally occurring wild-type proteins, as well as functional subsequences, modified forms or sequence variants so long as the subsequence, modified form or variant retain some degree of functionality of the native full-length protein. For example, a protein can have a deletion, substitution or addition and retain at least partial function or activity.

The terms “modify” or “variant” and grammatical variations thereof mean that a nucleic acid or polypeptide deviates from a reference sequence. Modified and variant sequences may therefore have substantially the same, greater or less expression, activity or function than a reference sequence, but at least retain partial activity or function of the reference sequence.

Non-limiting examples of modifications include one or more nucleotide or amino acid substitutions (e.g., 1-3, 3-5, 5-10, 10-15, 15-20, 20-25, 25-30, 30-40, 40-50, 50-100, 100-150, 150-200, 200-250, 250-500, 500-750, 750-850 or more nucleotides or residues).

An example of an amino acid modification is a conservative amino acid substitution or a deletion (e.g., subsequences or fragments) of a reference sequence. In particular embodiments, a modified or variant sequence retains at least part of a function or activity of unmodified sequence.

All mammalian and non-mammalian forms of nucleic acids that are transcribed and nucleic acids that encode proteins are included. Thus, the invention includes genes and proteins from non-mammals, mammals other than humans, and humans, which genes and proteins function in a substantially similar manner to human genes and proteins.

Following production of recombinant viral (e.g., AAV) vectors as set forth herein, if desired the viral (e.g., rAAV) virions can be purified and/or isolated from host cells using a variety of conventional methods. Such methods include column chromatography, CsCI gradients, and the like. For example, a plurality of column purification steps such as purification over an anion exchange column, an affinity column and/or a cation exchange column can be used. (See, e.g., International Publication No. WO 02/12455 and US Application Publication Nos. 20030207439). Alternatively, or in addition, CsCl gradient steps can be used. (See, e.g., US Application Publication Nos. 20120135515; and 20130072548) Further, if the use of infectious virus is employed to express the packaging and/or helper proteins, residual virus can be inactivated, using various methods. For example, adenovirus can be inactivated by heating to temperatures of approximately 60° C. for, e.g., 20 minutes or more. This treatment effectively inactivates the helper virus since AAV is heat stable while the helper adenovirus is heat labile.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more contaminants such as protein, nucleic acid, lipid, carbohydrate, cell membrane.

With respect to RNA molecules, the term “isolated” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule. Alternatively, this term may refer to a protein which has been sufficiently separated from other proteins with which it would naturally be associated, so as to exist in “substantially pure” form.

The term “isolated” does not exclude combinations produced by the hand of man, for example, a recombinant vector (e.g., rAAV) sequence, or virus particle that packages or encapsidates a vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or about 90-99% by weight, of the compound of interest. Purity is measured by methods appropriate for the compound of interest (e.g. chromatographic methods, agarose or polyacrylamide gel electrophoresis, HPLC analysis, and the like).

Nucleic acid molecules, expression vectors (e.g., vector genomes), plasmids, may be prepared by using recombinant DNA technology methods. The availability of nucleotide sequence information enables preparation of isolated nucleic acid molecules by a variety of means. For example, nucleic acids (e.g., plasmids) can be made using various standard cloning, recombinant DNA technology, via cell expression or in vitro translation and chemical synthesis techniques. Purity can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids may be maintained as DNA in any convenient cloning vector. In one embodiment, nucleic acids are maintained in a plasmid. Alternatively, nucleic acids may be maintained in vector suitable for expression in mammalian cells.

Invention nucleic acids, vectors, expression vectors (e.g., rAAV), and recombinant virus particles, methods and uses permit the treatment of genetic diseases. For deficiency state diseases, gene transfer can be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. The use of site-specific integration of nucleic acid sequences to correct defects is also possible.

Viral vectors such as lenti- and parvo-virus vectors, including AAV serotypes and variants thereof provide a means for delivery of nucleic acid into cells ex vivo, in vitro and in vivo, which encode proteins such that the cells express the encoded protein. AAV are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example. Because AAV are not associated with pathogenic disease in humans, AAV vectors are able to deliver heterologous polynucleotide sequences (e.g., therapeutic proteins and agents) to human patients without causing substantial AAV pathogenesis or disease.

Viral vectors which may be used include, but are not limited to, adeno-associated virus (AAV) vectors of multiple serotypes (e.g., AAV-1 to AAV-12, and others) and hybrid/chimeric AAV vectors, lentivirus vectors and pseudo-typed lentivirus vectors (e.g., Ebola virus, vesicular stomatitis virus (VSV), and feline immunodeficiency virus (FIV)), herpes simplex virus vectors, adenoviral vectors (with or without tissue specific promoters/enhancers), vaccinia virus vectors, retroviral vectors, lentiviral vectors, non-viral vectors and others.

AAV and lentiviral particles may be used to advantage as vehicles for effective gene delivery. Such virions possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells. Non-viral vectors, for example, based on plasmid DNA or minicircles, are also suitable gene transfer vectors.

Accordingly, in various embodiments of the invention a vector includes a lenti- or parvo-viral vector, such as an adeno-viral vector. In particular embodiments, a recombinant vector is a parvovirus vector. Parvoviruses are small viruses with a single-stranded DNA genome. “Adeno-associated viruses” (AAV) are in the parvovirus family.

AAV vectors and lentiviral vectors do not typically include viral genes associated with pathogenesis. Such vectors typically have one or more of the wild type AAV genes deleted in whole or in part, for example, rep and/or cap genes, but retain at least one functional flanking ITR sequence, as necessary for the rescue, replication, and packaging of the recombinant vector into an AAV vector particle. For example, only the essential parts of vector e.g., the ITR and LTR elements, respectively are included. An AAV vector genome would therefore include sequences required in cis for replication and packaging (e.g., functional ITR sequences).

Recombinant AAV vector, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, or AAV-2i8, for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be a AAV1, AAV3, AAV4, AAV5, AAV6, AAV7, AAV8, AAV9, AAV10, AAV11, AAV12, or AAV-2i8 or variant thereof, for example. AAV variants include variants and chimeras of AAV1, AAV2, AAV3, AAV4, AAV5, AAV6, AAV7, AAV8, AAV9, AAV10, AAV11, AAV12 and AAV-2i8 capsids.

In particular embodiments, adeno-associated virus (AAV) vectors include AAV1, AAV2, AAV3, AAV4, AAV5, AAV6, AAV7, AAV8, AAV9, AAV10, AAV11, AAV12, and AAV-2i8, as well as variants (e.g., capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773, discloses LK01, LK02, LK03, etc.).

AAV and AAV variants (e.g., capsid variants) serotypes (e.g., VP1, VP2, and/or VP3 sequences) may or may not be distinct from other AAV serotypes, including, for example, AAV1-AAV12 (e.g., distinct from VP1, VP2, and/or VP3 sequences of any of AAV1-AAV12 serotypes).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

In various exemplary embodiments, an AAV vector related to a reference serotype has a polynucleotide, polypeptide or subsequence thereof that includes or consists of a sequence at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, or AAV-2i8 (e.g., such as an ITR, or a VP1, VP2, and/or VP3 sequences).

Compositions, methods and uses of the invention include AAV sequences (polypeptides and nucleotides), and subsequences thereof that exhibit less than 100% sequence identity to a reference AAV serotype such as AAV1, AAV2, AAV3, AAV4, AAV5, AAV6, AAV7, AAV8, AAV9, AAV10, AAV11, AAV12, or AAV-2i8, but are distinct from and not identical to known AAV genes or proteins, such as AAV1, AAV2, AAV3, AAV4, AAV5, AAV6, AAV7, AAV8, AAV9, AAV10, AAV11, AAV12, or AAV-2i8, genes or proteins, etc. In one embodiment, an AAV polypeptide or subsequence thereof includes or consists of a sequence at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to any reference AAV sequence or subsequence thereof, such as AAV1, AAV2, AAV3, AAV4, AAV5, AAV6, AAV7, AAV8, AAV9, AAV10, AAV11, AAV12, or AAV-2i8 (e.g., VP1, VP2 and/or VP3 capsid or ITR). In particular aspects, an AAV variant has 1, 2, 3, 4, 5, 5-10, 10-15, 15-20 or more amino acid substitutions.

Recombinant AAV vectors, including AAV1, AAV2, AAV3, AAV4, AAV5, AAV6, AAV7, AAV8, AAV9, AAV10, AAV11, AAV12 or AAV-2i8 and variant, related, hybrid and chimeric sequences, can be constructed using recombinant techniques that are known to the skilled artisan, to include one or more nucleic acid sequences (transgenes) flanked with one or more functional AAV ITR sequences.

Nucleic acids (plasmids), vectors, recombinant vectors (e.g., rAAV), and recombinant virus particles can be incorporated into pharmaceutical compositions. Such pharmaceutical compositions are useful for, among other things, administration and delivery to a subject in vivo or ex vivo. In particular embodiments, pharmaceutical compositions contains a pharmaceutically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery. Thus, pharmaceutical compositions include carriers, diluents, or excipients suitable for administration by various routes.

Compositions and methods may be sterile. The compositions may be made and methods may be performed in containers suitable for such processes. Such containers include dishes, flasks, roller bottles, bags, bioreactors, vessels, tubes, vials, etc. Containers may be made of materials that include but are not limited to glass, plastic and polymers, such as polystyrene, polybutylene, polypropylene, etc.

The compositions and method steps may be performed in a designated order, or rearranged order. The method steps can be performed in stages or at intervals with intervening time periods. In other words, a method step can be performed, and then an interval of time between the next step can occur, such intervals ranging, for example, from about 1 second to about 60 seconds; from about 1 minute to about 60 minutes; from about 1 hour to about 24 hours; from about 1 day to about 7 days; or from about 1 week to about 48 weeks.

Protocols for the generation of adenoviral vectors have been described in U.S. Pat. Nos. 5,998,205; 6,228,646; 6,093,699; and 6,100,242; and International Patent Application Nos. WO 94/17810 and WO 94/23744, which are incorporated herein by reference in their entirety.

The invention is useful in producing cells and vectors for human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases such as HemA and others known to those of skill in the art.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Recombinant vector (e.g., rAAV) sequences, recombinant virus particles, and pharmaceutical compositions thereof can be packaged in single or multiple unit dosage form for ease of administration and uniformity of dosage.

The invention provides kits with packaging material and one or more components therein. A kit typically includes a label or packaging insert including a description of the components or instructions for use of the components therein. A kit can contain a collection of such components, e.g., a nucleic acid (plasmid), PEI, cells.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein. Labels or inserts can include information identifying manufacturer, lot numbers, manufacture location and date, expiration dates. Labels or inserts can include information identifying manufacturer information, lot numbers, manufacturer location and date. Labels or inserts can include instructions for using one or more of the kit components in a method, use, or manufacturing protocol. Instructions can include instructions for producing the compositions or practicing any of the methods described herein.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, patent applications, publications, and other references, GenBank citations and ATCC citations cited herein are incorporated by reference in their entirety. In case of conflict, the specification, including definitions, will control.

Various terms relating to the biological molecules of the invention are used hereinabove and also throughout the specification and claims.

All of the features disclosed herein may be combined in any combination. Each feature disclosed in the specification may be replaced by an alternative feature serving a same, equivalent, or similar purpose. Thus, unless expressly stated otherwise, disclosed features (e.g., PEI, plasmid, vector (e.g., rAAV, or recombinant virus particle) are an example of a genus of equivalent or similar features.

As used herein, the singular forms “a”, “and,” and “the” include plural referents unless the context clearly indicates otherwise. Thus, for example, reference to “a plasmid” or “a nucleic acid” includes a plurality of such plasmids or nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94%, etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### Example 1

This example includes a description of various materials and methods.

Cell Culture: FreeStyle™293F (293F) cells purchased from Thermo Fisher Scientific (Invitrogen™ by Thermo Fisher Scientific, R790-07) were cultured in FreeStyle™ F17 (F17) expression medium (Gibco cat no. A13835-01) or FreeStyle™ 293 (FS) Expression medium (Gibco cat no. 12338-018) supplemented with 4 mM GlutaMAX (Life Technologies, cat. no. 35050-061). Cells were cultured in variety cell culture apparatus, including spinner flasks and bioreactors. For spinner flask culture (Bellco Glass, cat. 1965-83100 or Corning, cat. 3152), cells were cultured at 37° C. incubator with 70 rpm agitation and a humidified atmosphere of 8% CO2; for bioreactors ((DASGIP Parallel Bioreactor system, Eppendorf), cell culture were controlled by programmed parameters, (DO 30%, pH7.2, 170 or 150 rpm). Typically, Cells were seeded at 0.25-0.5×10⁶/mL, subcultured every 2-3 days when cell density reached approximately 1.6-2×10⁶/mL by adding fresh cell culture media. Cell density and viability were determined using a hemacytometer after Trypan Blue staining.

Plasmids: Three plasmids were used to produce recombinant adeno-associated viral vectors (rAAV): 1) Transgene plasmid containing eGFP flanked by ITRs; 2) a packaging plasmid containing AAV serotype 2 rep and cap genes; and 3) an adenoviral helper plasmid containing adenovirus E2, E4 and VARNA genes. All plasmids were purchased from and manufactured by Aldevron. P

Preparation of PEI solutions: Linear polyethylenimine (PEI) 25 KDa (Polysciences, Cat. No. 23966-2), PEI “Max” 40 KDa ((Polysciences, Cat. No. 24765-2, hydrochloride salt of the linear PEI 25 KDa) and PEIpro (Polyplus, cat. 115-010) were used as transfection reagents. For most transfection studies, PEI “Max” was dissolved in 5 mM Tris to make a 0.5 mg/mL solution and pH was adjusted to 7.10. PEI 25 KDa was first dissolved in 80° C. hot water, after cooling down, Tris buffer was added to make 0.5 mg/mL of PEI in 5 mM Tris buffer solution, pH7.10. For some studies in which PEI “Max” 40 KDa was compared to PEI 25 KDa on transfection, PEI 40 KDa and 25 KDa was either dissolved in 5 mM Tris buffer with or without 150 mM NaCl or in water with or without 150 mM NaCl, adjust pH to 7.10.

Transfection: 293F cells were grown in either FS medium or F17 medium plus 4 mM GlutaMAX™ Supplement in spinner flasks. One day before transfection, cells were subcultured by adding fresh media, on the day of transfection, cells density was determined using hemacytometer and further diluted with fresh FS media or F17 media to final density of 0.35-1×10⁶ cells/mL, transfection was performed either in suspension cell culture wells or spinner flasks.

Three plasmids as described above were used in transfection at molar ratio 1:1:1. Total DNA amount used for transfection was from 0.7 to 11.2 μg per ml of cell culture. PEI/DNA complex was prepared with different ratio of PEI and DNA, incubated at room temperature from 1 min to up to 30 min, then the DNA/PEI complexes were added dropwise to the cell culture. To evaluate the effect of Free PEI on transfection efficiency and rAAV productivity, PEI molecules were prepared the same way as described above without incubation with DNA and added to the cell culture immediately after DNA/PEI complexes was added. Samples, including cells and cell culture media, were taken at 24, 48 and 72 hr post transfection for transfection efficiency and other assays and cells culture was harvested at 72 h post transfection.

Transfection efficiency was assessed either using an inverted fluorescence microscope (Leica) or a flow cytometer (Becton Dickinson Biosciences). eGFP positive cells were detected using fluorescence microscope. The percentage of GFP positive cells was assessed using a BD FACS Canto flow cytometer.

Production of rAAV vectors in bioreactors: A 2 L DASGIP Parallel Bioreactor system (Eppendorf) equipped with two pitched blade impellers was used to scale up the vector production process. The final working volume was adjusted to 400 mL in the studies. The agitation was set to 150 rpm or 170 rpm, the temperature was maintained at 37° C. and pH 7.2 during cell culture. Dissolved oxygen was maintained at 30% by supplementation with a gas mix of oxygen, carbon dioxide and air. All these parameters were monitored and controlled by DASGIP Control System with DASGIP Control 4.0 software. 293F cells cultured in F17 medium were inoculated at a cell density 0.4×10⁶ cells/mL with viability greater than 95%. Cell were subculture at day 2 or day 3 after seeding by adding fresh medium, cell density was approximately 0.4-0.7×10⁶ cells/mL after subculture. Twenty-four hours post subculture, cells were transfected with PEI/DNA complex as described in the legends, and the cell density is approximately 1×10⁶ cells/mL at transfection. PEI/DNA weight ratio 2:1 with ½ of PEI as Free PEI at transfection. Samples were collected every 24 h up to 72 h.

Quantitation of rAAV vectors: rAAV vectors were released from the transfected 293F cell harvest by either passing frozen/thawed three cycles or Microfluidizer™ (Microfluidics) three times. The cell debris was pelleted by centrifugation and the supernatants were collected for real-time PCR and transduction assays.

AAV vector genome copy number was determined with real-time polymerase chain reaction (Q-PCR) (QuanStudio 7, Life Technologies) using TaqMan Master Mix (cat. 4304437, Life technologies). 10 μL of cell lysate was treated with 7.6 U DNase I (Cat#79254, Qiagen) to digest contaminating unpackaged DNA, and then treated with 0.2% SDS/5 mM EDTA/0.2M NaCl at 95° C. for 10 min to inactivate DNase I and release vector DNA. The primers and probe detected transgene eGFP sequence: Forward primer: 5′-GCACAAGCTGGAGTACAACTA-3′, reverse primer 5′-TGTTGTGGCGGATCTTGAA-3′ and probe 5′-/56-FAM/AGCAGAAGA/ZEN/ACGGCATCAAGGTGA/3IABkFQ/-3′. To generate a standard curve, a pAAV-eGFP-WRPE plasmid was linearized by HindIII-HF digestion and used in 1:5 serial dilutions from 1×10⁸ to 1.28×10³ gene copies. All samples were performed in triplicate.

Transduction assays were performed by adding 50 μL cell lysates to HEK 293 cells seeded in 48-well plates. Etoposide was added to each well at the time of transduction to the final concentration of 3 uM. After 48 h incubation, GFP positive cells were assessed with an inverted fluorescence microscope.

### Example 2

This example includes a description of various results.

Effect of transfection medium on transfection efficiency and rAAV production: To develop a scalable serum-free suspension culture system to produce rAAV vector at large scale, several media suitable for suspension cell culture, including FS medium, F17 medium, SFM4Transfx-293 and others, were evaluated to grow 293F cells. The studies reveal that FS 293 medium and F17 medium support good cell growth—cell density reached 2.5-3×10⁶ cells/mL in spinner flask. These two media also support efficient gene transfection into 293F cells.

Effect of Free PEI on transfection efficiency and rAAV production: High transfection efficiency is a step towards achieving high rAAV production. Polyethylenimine (PEI), as a transfection reagent, was employed to transfect 293F cells in suspension culture.

Among the numerous parameters evaluated, the molar ratio of nitrogen (N) in PEI molecule to the phosphate (P) of DNA (N:P ratio) affects the transfection efficiency dramatically, from very poor transfection to highly efficient transfection when the N:P ratio was changed from low to high. Cytotoxicity was also found at high N:P ratio, such as N:P ratio 30, when used for the transfection.

Several different PEI molecules were studied including PEI “Max” 40 KDa, PEI 25 KDa, PEIPro and others. PEI molecules were prepared either using Tris buffer or DI water with or without 150 mM NaCl, at pH 7.1, appropriate amount of plasmid DNA was mixed with PEI molecules at fixed N:P ratio, incubated at room temperature for different time periods, such as 1 minutes, 5 minutes, 10 minutes, 15 minutes, 20 minutes and up to 30 minutes, and then used to transfect cells.

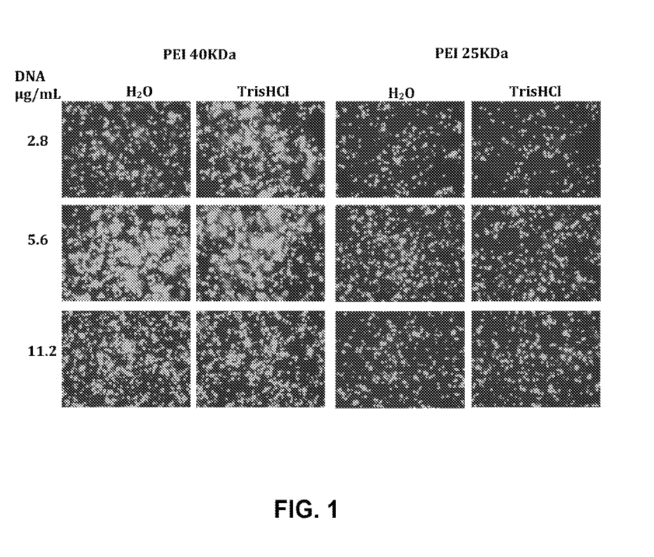

The data show that both PEI “Max” 40 KDa and PEI 25 KDa provided high efficiency cell transfection, with PEI “Max” 40 KDa providing consistently high transfection efficiency (FIG. 1). Thus, most data were generated using PEI “Max” 40 KDa as transfection reagent.

RAAV vectors were produced by transfection of 293F cells using three plasmids as described in the materials and methods. Different cell culture apparatus including 12-well plate, cell culture spinner flask and bioreactor were used to culture 293F cells and produce rAAV vectors. PEI/DNA mixture was prepared at a weight ratio of 2:1 and 4:1 to transfect the cells. Different DNA amounts per ml cell culture were tested for transfection efficiency and a molar ratio of 1:1:1 among the three plasmids were typically used for transfection. 293F cells were cultured in serum-free suspension culture using FS medium and F17 medium.

PEI mediated gene transfer is a very complicated cell biological process, involving binding to the cell receptors, endocytosis, intracellular trafficking, nuclei entry and gene expression are only a few of the key steps. While not wishing to be bound by any theory, an appropriate amount of Free PEI molecules may enhance transfection efficiency and in turn increase rAAV production.

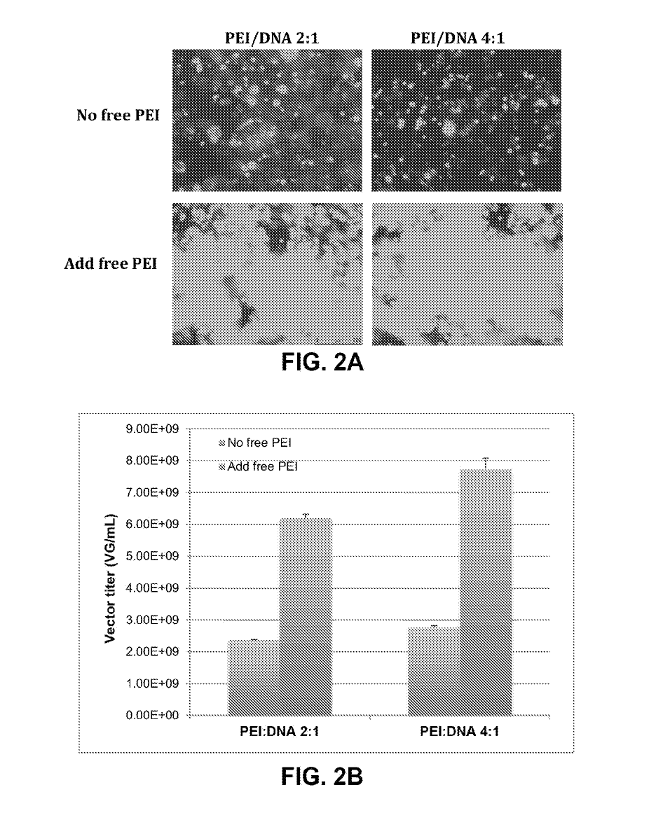

As indicated in FIG. 2A, cell transfection efficiency was significantly improved when Free PEI molecules were added to the cell culture right after addition of DNA/PEI complex to the cell culture, as compared to the transfection efficiency with DNA/PEI complex only. This phenomena, Free PEI boosted PEI transfection efficiency, was observed in 12-well cell culture plates (FIG. 2A), cell culture Spinner flasks and bioreactor. In addition, rAAV vector production correspondingly increased when Free PEI molecules were added in the transfection-2 to 3 fold more rAAV vectors produced (FIG. 2B).

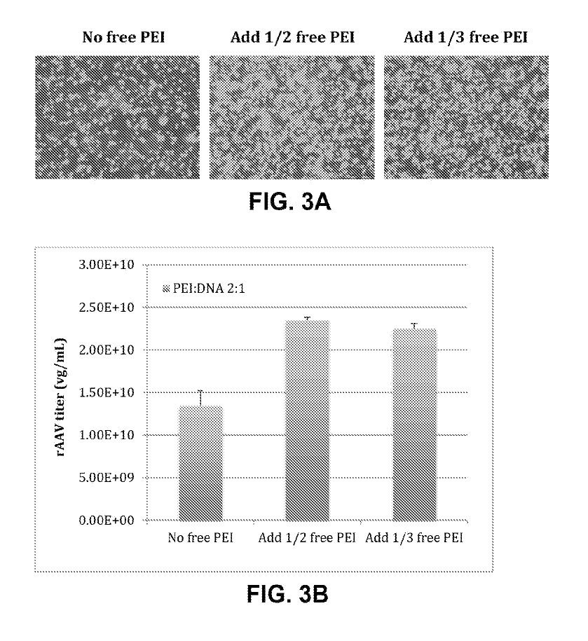

The discovery, that Free PEI enhances transfection efficiency and rAAV production, was further tested in larger cell culture scales, spinner flasks and bioreactors. The transfection results in Spinner flasks with or without Free PEI (FIG. 3) are consistent with what was found in 12-well plates, namely, that transfection efficiency was significantly enhanced when Free PEI was used in the transfection and rAAV productivity increased 2 to 3 fold, from 1.3E+10 vector genome (VG) per ml of cell lysate without Free PEI to about 2.4E+10 VG/mL with Free PEI.

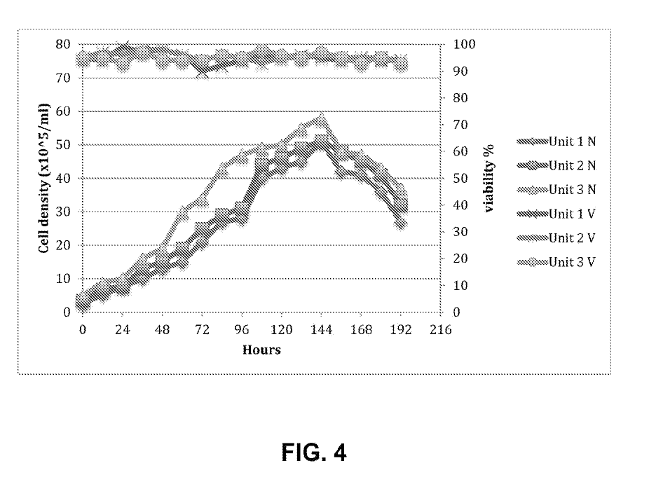

Cell growth status at transfection also had a significant impact on rAAV vector production in Bioreactor. To further evaluate ways to improve rAAV production, cell growth status was tested to determine impact on rAAV vector yield. In suspension cell culture, cells were seeded at 0.25×10⁶ cells/mL, 0.35×10⁶ cells/mL and 0.5×10⁶ cells/mL, and a cell growth curve was established over 7 days of cell culture (FIG. 4) in a 2 L bioreactor. The cell growth phases were roughly defined and the exponential growth phase was determined in between 48 h to 84 h. The peak of cell density was 4.8-5.8×10⁶ cells/mL at day 6 and then cell density started to drop. Cell viability was above 90% during the culture.

For transfection and rAAV production, 293F cells were inoculated at a cell density 0.4×10⁶ cells/mL with viability greater than 95% in the 2 L bioreactor with working volumes about 400 ml. While trying to identify the best cell culture window(s) for plasmid transfection and rAAV production, it was discovered that transfection of cells at different cell culture status, also has a significant effect on rAAV yield.

Cells were then subcultured at either day 2 or days 3 post cell seeding by adding fresh cell culture media to reduce cell density, cell densities are in the range of at 0.4-0.7×10⁶ cells/mL. Cells were then transfected 24 hours later after subculture using PEI mediated transfection method as described. Typically, cell densities at transfection will achieve 7E+05 cell/ml to 1.3E+06 cells/ml of media. The conditions of two independent experiments and corresponding results, are shown in Table 1.

TABLE 1
rAAV vector production in bioreactor

| | Unit 1 | Unit 4 | Unit 2 | Unit 3 |
|---|---|---|---|---|
| Cell/medium | 293F/F17 | 293F/F17 | 293F/F17 | 293F/F17 |
| Seeding cell density | 4E+05/mL | 4E+05/mL | 4E+05/mL | 4E+05/mL |
| Agitation | 170 rpm | 150 rpm | 170 rpm | 150 rpm |
| Subculture | Day 3 | Day 3 | Day 2 | Day 2 |
| Cell density after subculture | 0.6 × 10⁶/mL | 0.5 × 10⁶/mL | 0.4-0.7 × 10⁶/mL | 0.5 × 10⁶/mL |
| Transfection (TF) | Day 4 | Day 4 | Day 3 | Day 3 |
| DNA μg/mL | 4.2 | 4.2 | 4.2 | 4.2 |
| Cell density at TF | 1 × 10⁶/mL | 0.9-1.3 × 10⁶/mL | 0.7-1 × 10⁶/mL | 0.7-1 × 10⁶/mL |
| Vector titer (vg/mL) | | | | |
| Experiment 1 | 5.11E+10 | 5.73E+10 | 7.72E+09 | 1.05E+10 |
| Experiment 2 | 8.37E+10 | 1.38E+11 | 7.00E+10 | 7.30E+10 |

It appears that subculture cells at day 3 post seeding and transfect the cells at day 4 post seed resulted in significantly higher rAAV productivity comparing to cell subculture at day 2 post seeding and transfected at day 3 post seeding

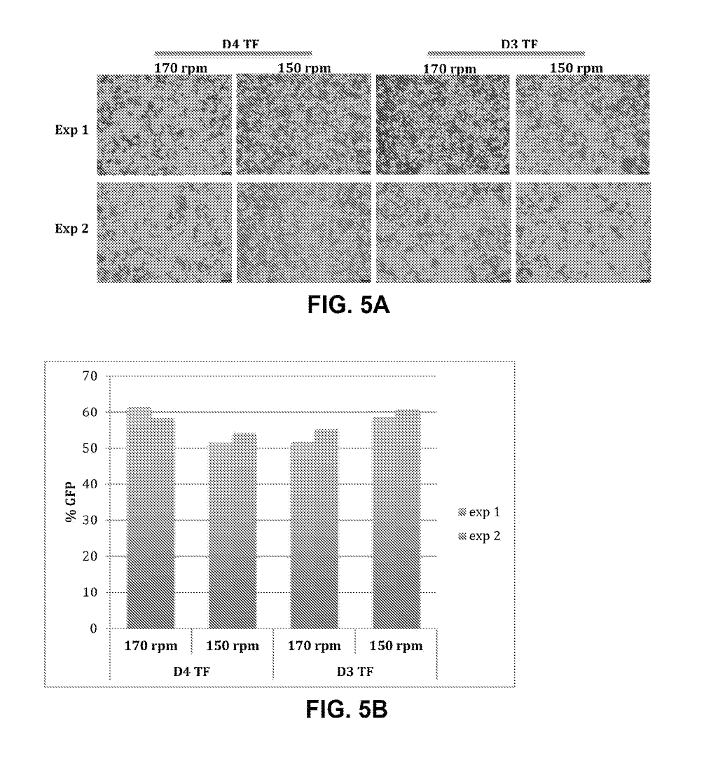

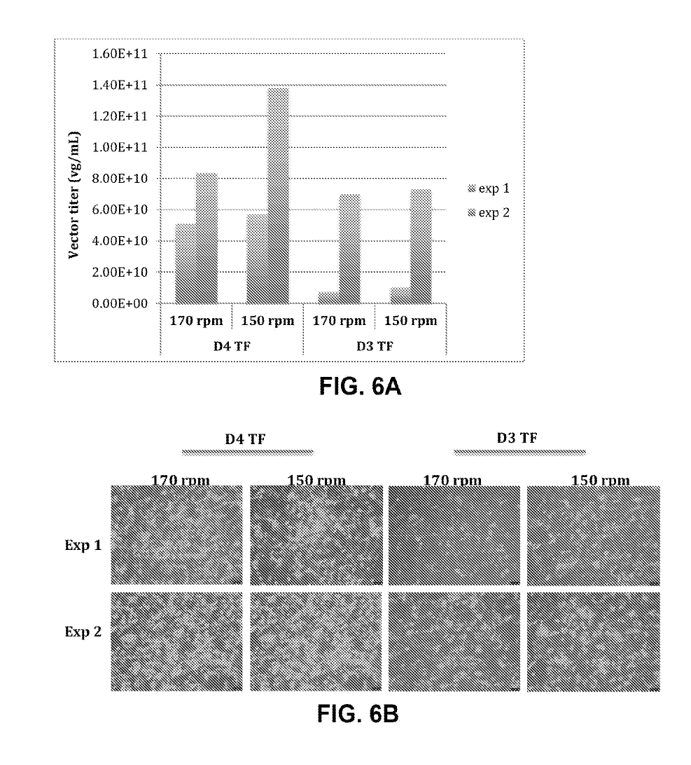

It is interesting to note that the transfection efficiency does not appear different between these two compared conditions, as indicated by the data in FIG. 5. Shown in FIG. 5A is a comparison of transfection efficiency as indicated by eGFP gene expression and FIG. 5B is more quantitative FACS data, suggesting the about 50% cells transfected at all the tested conditions; however, the vector titer was significantly higher on day 4 transfection assessed by qPCR (FIG. 6A), 2 fold higher, 5 fold higher, sometime even 7 to 8 fold higher in the day 4 transfection condition in comparing with day 3 transfection. The highest titer was 1.38E+11 vg/mL in the condition of transfection at day 4 and agitation speed at 150 rpm.

Transduction function of rAAV-GFP from these experiments was assessed by transduction of HEK 293 cell, consistent with qPCR analysis, when same volume of cell lysate from each production condition were added to HEK 293 cells, higher transduction was observed from the samples of day 4 transfection in comparing with the sample of day 3 transfection (FIG. 6B). These data indicate that cell growth stage and metabolic status may play an important role in rAAV production, and that there may be cell metabolic window that is more permeable for rAAV biosynthesis, in addition transfection efficiency.

The newly developed PEI mediate rAAV production system is a fully scalable, cGMP compliant, versatile rAAV production platform, which can be used to produce any serotypes of rAAV vectors in serum-free suspension cell culture. In combination with PEI as transfection reagent, such as high efficient PEI “Max” 40 KDa molecules, addition of Free PEI into the transfection process and a discovered primed cell growth stage for transfection, very high rAAV productivity in suspension cell culture conditions was achieved, which is about 10 fold higher than in the similar serum free suspension culture systems reported in the literature (summarized in Table 2).

TABLE 2
Comparison of vector yield from serum free suspension cell culture
methods with that reported in the literature (reference)

| Serotypes | Titer (vg/mL) | Reference |
|---|---|---|
| AAV2-GFP | 1.2 × 10¹⁰ | Parminder Singh Chahal et al. (2014) |
| AAV6-GFP | 2.0 × 10⁹ | Parminder Singh Chahal et al. (2014) |
| AAV2-GFP | 3.1 × 10¹⁰ | Yves Durocher et al. (2007) |
| AAV2-GFP | 3.2 × 10⁹ | Yves Durocher et al. (2007) |
| AAV2-GFP | 1.1 × 10¹⁰ | Hildinger M et al. (2007)* |
| AAV5-GFP | 1.6 × 10¹⁰ | Hildinger M et al. (2007)* |
| AAV2-GFP | 1-2 × 10¹¹ | Spark P D |

While certain of the embodiments of the invention have been described and specifically exemplified above, it is not intended that the invention be limited to such embodiments. Various modifications may be made thereto without departing from the scope and spirit of the invention, as set forth in the following claims.

1. A composition comprising a plasmid/PEI mixture, said plasmid/PEI mixture comprising components:
(a) one or more plasmids comprising nucleic acids encoding AAV packaging proteins and/or nucleic acids encoding helper proteins;
(b) a plasmid comprising a nucleic acid that encodes a protein or is transcribed into a transcript of interest; and
(c) a polyethylenimine (PEI) solution,
wherein said plasmids are in a molar ratio range of about 1:0.01 to about 1:100, or are in a molar ratio range of about 100:1 to about 1:0.01, and wherein the mixture of said components (a), (b) and (c) has optionally been incubated for a period of time from about 10 seconds to about 4 hours.

2. A composition according to claim 1, further comprising cells.

3. A composition according to claim 2, wherein said cells are in contact with said plasmid/PEI mixture of said components (a), (b) and (c).

4. A composition according to claim 3, further comprising Free PEI.

5. A composition according to claim 4, wherein said cells are in contact with said Free PEI.

6. A composition according to claim 3, wherein said cells have been in contact with the mixture of said components (a), (b) and (c) for at least about 4 hours.

7.-8. (canceled)

9. A composition according to claim 5, wherein said cells have been in contact with the mixture of said components (a), (b) and (c) and said Free PEI for at least about 4 hours.

10.-11. (canceled)

12. A composition according to claim 6, wherein said cells produce recombinant AAV vector comprising the nucleic acid that encodes the protein or is transcribed into a transcript of interest.

13. A composition according to claim 1, wherein said composition comprises a container, said container optionally comprising a flask, plate, bag, or bioreactor, said container optionally sterile, and/or said container optionally suitable for maintaining cell viability or growth.

14. A composition according to claim 1, further comprising a plasmid comprising nucleic acids that encode AAV capsid proteins.

15. A composition according to claim 14, further comprising cells in contact with said plasmid/PEI mixture of said components (a), (b) and (c) and said plasmid comprising nucleic acids that encode AAV capsid proteins.

16. A composition according to claim 15, further comprising Free PEI, wherein said cells are in contact with said Free PEI.

17. A composition according to claim 16, wherein said cells have been in contact with the mixture of said components (a), (b) and (c), said plasmid comprising nucleic acids that encode AAV capsid proteins, and said Free PEI for at least about 4 hours.

18.-19. (canceled)

20. A method for producing transfected cells, said method comprising:
(a) providing a plasmid;
(b) providing a solution comprising polyethylenimine (PEI);
(c) mixing the plasmid of (a) with the PEI solution of (b), to produce a plasmid/PEI mixture, and optionally incubating said plasmid/PEI mixture for a period in the range of about 10 seconds to about 4 hours;
(d) contacting cells with said plasmid/PEI mixture of step (c), to produce a plasmid/PEI cell culture;
(e) adding Free PEI to said nucleic acid/PEI cell culture produced in step (d) to produce a Free PEI/plasmid/PEI cell culture; and
(f) incubating said Free PEI/plasmid/PEI cell culture of step (e) for at least about 4 hours, thereby producing transfected cells.

21. A method according to claim 20, wherein said plasmid comprises a nucleic acid that encodes a protein or is transcribed into a transcript of interest.

22. A method for producing transfected cells that produce recombinant AAV vector comprising a nucleic acid that encodes a protein or is transcribed into a transcript of interest, said method comprising:
(a) providing one or more plasmids comprising nucleic acids encoding AAV packaging proteins and/or nucleic acids encoding helper proteins;
(b) providing a plasmid comprising a nucleic acid that encodes a protein or is transcribed into a transcript of interest;
(c) providing a solution comprising polyethylenimine (PEI);
(d) mixing the plasmids of steps (a) and (b) with the PEI solution of step (c), wherein said plasmids are in a molar ratio range of about 1:0.01 to about 1:100, or are in a molar ratio range of about 100:1 to about 1:0.01, to produce a plasmid/PEI mixture, and optionally incubating said plasmid/PEI mixture for a period in the range of about 10 seconds to about 4 hours;
(e) contacting cells with said plasmid/PEI mixture of step (d), to produce a plasmid/PEI cell culture;
(f) adding Free PEI to said plasmid/PEI cell culture produced in step (e) to produce a Free PEI/plasmid/PEI cell culture; and
(g) incubating said Free PEI/plasmid/PEI cell culture of step (f) for at least about 4 hours, thereby producing transfected cells that produce recombinant AAV vector comprising a nucleic acid that encodes a protein or is transcribed into a transcript of interest.

23. A method according to claim 20, further comprising the step of harvesting said transfected cells produced in steps (f) or (g) and/or culture medium from said transfected cells produced in steps (f) or (g) to produce a cell and/or culture medium harvest.

24. A method according to claim 22, further comprising isolating and/or purifying recombinant AAV vector from said cell and/or culture medium harvest of step (g) thereby producing recombinant AAV vector comprising a nucleic acid that encodes a protein or is transcribed into a transcript of interest.

25. A method for producing recombinant AAV vector comprising a nucleic acid that encodes a protein or is transcribed into a transcript of interest, said method comprising the steps:
(a) providing one or more plasmids comprising nucleic acids encoding AAV packaging proteins and/or nucleic acids encoding helper proteins;
(b) providing a plasmid comprising a nucleic acid that encodes a protein or is transcribed into a transcript of interest;
(c) providing a solution comprising polyethylenimine (PEI);
(d) mixing said plasmids of steps (a) and (b) with said PEI solution of step (c), wherein said plasmids are in a molar ratio range of about 1:0.01 to about 1:100, or are in a molar ratio range of about 100:1 to about 1:0.01, to produce a plasmid/PEI mixture, and optionally incubating the plasmid/PEI mixture for a period of time in the range of about 10 seconds to about 4 hours;
(e) contacting cells with said plasmid/PEI mixture produced in step (d), to produce a plasmid/PEI cell culture;
(f) adding Free PEI to said plasmid/PEI cell culture produced in step (e) to produce a Free PEI/plasmid/PEI cell culture;
(g) incubating said plasmid/PEI cell culture of step (e) or said Free PEI/plasmid/PEI cell culture of step (f) for at least about 4 hours to produce transfected cells;
(h) harvesting said transfected cells produced in step (g) and/or culture medium from the transfected cells produced in step (g) to produce a cell and/or culture medium harvest; and
(i) isolating and/or purifying recombinant AAV vector from said cell and/or culture medium harvest produced in step (h) thereby producing recombinant AAV vector comprising a nucleic acid that encodes a protein or is transcribed into a transcript of interest.

26. A method for producing transfected cells that produce recombinant AAV vector comprising a nucleic acid that encodes a protein or is transcribed into a transcript of interest, said method comprising the steps:
(a) providing a mixture of components (i), (ii) and (iii):
(i) one or more plasmids comprising nucleic acids encoding AAV packaging proteins and/or nucleic acids encoding helper proteins;
(ii) a plasmid comprising a nucleic acid that encodes a protein or is transcribed into a transcript of interest; and
(iii) a polyethylenimine (PEI) solution,
(b) mixing said plasmids (i) and (ii) with said PEI solution (iii) so that said plasmids are in a molar ratio range of about 1:0.01 to about 1:100, or in a molar ratio range of about 100:1 to about 1:0.01, to produce a plasmid/PEI mixture, and optionally incubating said plasmid/PEI mixture for a period of time in the range of about 10 seconds to about 4 hours;
(c) contacting cells with said plasmid/PEI mixture produced in step (b) to produce a plasmid/PEI cell culture;
(d) adding Free PEI to said plasmid/PEI cell culture produced in step (c) to produce a Free PEI/plasmid/PEI cell culture;
(e) incubating said plasmid/PEI cell culture of step (c) or said Free PEI/plasmid/PEI cell culture of step (d) for at least about 4 hours to produce transfected cells that produce recombinant AAV vector comprising a nucleic acid that encodes a protein or is transcribed into a transcript of interest.

27. A method according to claim 25, further comprising the step of harvesting the transfected cells produced in step (e) and/or culture medium from the transfected cells produced in step (e) to produce a cell and/or culture medium harvest; and/or isolating and/or purifying recombinant AAV vector from the cell and/or culture medium harvest of step (e), thereby producing recombinant AAV vector comprising a nucleic acid that encodes a protein or is transcribed into a transcript of interest.

28. A method for producing recombinant AAV vector comprising a nucleic acid that encodes a protein or is transcribed into a transcript of interest, comprising the step:
(a) providing a mixture of components (i), (ii) and (iii):
(i) one or more plasmids comprising nucleic acids encoding AAV packaging proteins and/or nucleic acids encoding helper proteins;
(ii) a plasmid comprising a nucleic acid that encodes a protein or is transcribed into a transcript of interest; and
(iii) a polyethylenimine (PEI) solution,
(b) mixing said plasmids (i) and (ii) with said PEI solution (iii) so that said plasmids are in a molar ratio range of about 1:0.01 to about 1:100, or are in a molar ratio range of about 100:1 to about 1:0.01, to produce a plasmid/PEI mixture, and optionally incubating said plasmid/PEI mixture for a period of time from about 10 seconds to about 4 hours;
(c) contacting cells with said plasmid/PEI mixture produced in step (b) to produce a plasmid/PEI cell culture;
(d) adding Free PEI to said plasmid/PEI cell culture produced in step (c) to produce a Free PEI/plasmid/PEI cell culture;
(e) incubating said plasmid/PEI cell culture of step (c) or said Free PEI/plasmid/PEI cell culture of step (d) for at least about 4 hours to produce transfected cells;
(f) harvesting said transfected cells produced in step (e) and/or culture medium from the transfected cells produced in step (e) to produce a cell and/or culture medium harvest; and
(g) isolating and/or purifying recombinant AAV vector from the cell and/or culture medium harvest produced in step (f), thereby producing recombinant AAV vector comprising a nucleic acid that encodes a protein or is transcribed into a transcript of interest.

29. A method for producing recombinant AAV vector comprising a nucleic acid that encodes a protein or is transcribed into a transcript of interest, comprising the steps:
(a) providing a mixture of components (i), (ii) and (iii):
(i) one or more plasmids comprising nucleic acids encoding AAV packaging proteins and/or nucleic acids encoding helper proteins;
(ii) a plasmid comprising a nucleic acid that encodes a protein or is transcribed into a transcript of interest; and
(iii) a polyethylenimine (PEI) solution,
wherein said plasmids (i) and (ii) are in a molar ratio range of about 1:0.01 to about 1:100, or are in a molar ratio range of about 100:1 to about 1:0.01, and wherein the mixture of components (i), (ii) and (iii) has optionally been incubated for a period of time from about 10 seconds to about 4 hours.
(b) contacting cells with the mixture produced in step (a) to produce a plasmid/PEI cell culture;
(c) adding Free PEI to said plasmid/PEI cell culture produced in step (b) to produce a Free PEI/plasmid/PEI cell culture;
(d) incubating said plasmid/PEI cell culture of step (b) or said Free PEI/plasmid/PEI cell culture of step (c) for at least about 4 hours to produce transfected cells;
(e) harvesting said transfected cells produced in step (d) and/or culture medium from the transfected cells produced in step (d) to produce a cell and/or culture medium harvest; and
(f) isolating and/or purifying recombinant AAV vector from the cell and/or culture medium harvest produced in step (e), thereby producing recombinant AAV vector comprising a nucleic acid that encodes a protein or is transcribed into a transcript of interest.

30. A composition according to claim 3, wherein said plasmid/PEI cell culture, or said or said Free PEI/plasmid/PEI cell culture, or said nucleic acid/PEI cell culture is incubated for a period of time in the range of about 4 hours to about 140 hours.

31. (canceled)

32. A composition according to claim 1, wherein said plasmid/PEI mixture has a PEI:plasmid weight ratio in the range of about 0.1:1 to about 5:1, or has a PEI:plasmid weight ratio in the range of about 5:1 to about 0.1:1, or wherein said Free PEI/plasmid/PEI cell culture has a PEI:plasmid weight ratio in the range of about 0.1:1 to about 5:1, or has a PEI:plasmid weight ratio in the range of about 5:1 to about 0.1:1.

33. (canceled)

34. A composition according to claim 1, wherein the PEI of said plasmid/PEI mixture and/or the Free PEI comprises a hydrolyzed linear polyethylenimine.

35. A composition according to claim 1, wherein the PEI of said plasmid/PEI mixture and/or said Free PEI comprises a hydrolyzed linear polyethylenimine with a molecular weight in the range of about 4,000 to about 160,000 and/or in the range of about 2,500 to about 250,000 molecular weight in free base form.

36. (canceled)

37. A composition according to claim 1, wherein the molar ratio of nitrogen (N) in Total PEI to phosphate (P) in plasmid is in the range of about 1:1 to about 50:1 (N:P) in said Free PEI/plasmid/PEI cell culture.

38. (canceled)

39. A composition according to claim 1, wherein said plasmid/PEI mixture is incubated for a period of time in the range of about 30 seconds to about 4 hours.

40. (canceled)

41. A composition according to claim 1, wherein the amount of Free PEI is in the range of about 10% to about 90% of Total PEI.

42.-43. (canceled)

44. A composition according to claim 4, wherein said Free PEI is added to said cells before, at the same time as, or after said plasmid/PEI mixture is contacted with said cells.

45. A composition according to claim 2, wherein said cells are in suspension culture.

46. A composition according to claim 2, wherein said cells are grown or maintained in a serum-free culture medium.

47. A composition according to claim 2, wherein said cells are at a density in the range of about 1×10⁵ cells/mL to about 1×10⁸ cells/mL when contacted with said plasmid/PEI mixture and/or when contacted with said Free PEI.

48. A composition according to claim 2, wherein viability of said cells when contacted with said plasmid/PEI mixture or with said Free PEI is about 60% or greater than 60%, or wherein said cells are in log phase growth when contacted with said plasmid/PEI mixture.

49. (canceled)

50. A composition according to claim 1, wherein the encoded AAV packaging proteins comprise AAV rep and/or AAV cap.

51. (canceled)

52. A composition according to claim 1, wherein the encoded helper proteins comprise adenovirus E2 and/or E4, VARNA proteins, and/or non-AAV helper proteins

53. A composition according to claim 2, wherein said cells are mammalian cells.

54. A composition according to claim 2, wherein said cells are HEK 293E or HEK 293F cells.

55. A composition according to claim 2, wherein the total amount of plasmid comprising the nucleic acid that encodes a protein or is transcribed into a transcript of interest and the one or more plasmids comprising nucleic acids encoding AAV packaging proteins and/or nucleic acids encoding helper proteins is in the range of about 0.1 μg to about 15 μg per mL of cells.

56. A composition according to claim 1, wherein the molar ratio of the plasmid comprising the nucleic acid that encodes a protein or is transcribed into a transcript of interest to the one or more plasmids comprising nucleic acids encoding AAV packaging proteins and/or nucleic acids encoding helper proteins is in the range of about 1:5 to about 1:1, or is in the range of about 1:1 to about 5:1.

57. A composition according to claim 1, wherein said one or more plasmids comprises a first plasmid comprising the nucleic acids encoding AAV packaging proteins and a second plasmid comprising the nucleic acids encoding helper proteins.

58. A composition according to claim 57, wherein the molar ratio of the plasmid comprising the nucleic acid that encodes a protein or is transcribed into a transcript of interest to the first plasmid comprising the nucleic acids encoding AAV packaging proteins to the second plasmid comprising the nucleic acids encoding helper proteins is in the range of about 1-5:1:1, or 1:1-5:1, or 1:1:1-5.

59. (canceled)

60. A composition according to claim 1, wherein the AAV vector comprises an AAV serotype or an AAV pseudotype, wherein said AAV pseudotype comprises an AAV capsid serotype different from an ITR serotype.

61. A composition according to claim 1, wherein the AAV vector further comprises an intron, an expression control element, one or more adeno-associated virus (AAV) inverted terminal repeats (ITRs) and/or a filler polynucleotide sequence.

62. (canceled)

63. A composition according to claim 61, wherein the expression control element comprises a constitutive or regulatable control element, or a tissue-specific expression control element or promoter.

64. A composition according to claim 61, wherein the expression control element comprises an element that confers expression in liver.

65. (canceled)

66. A composition according to claim 1, wherein the AAV vector comprises a VP1, VP2 and/or VP3 capsid protein having 75% or more sequence identity to any of AAV1, AAV2, AAV3, AAV4, AAV5, AAV6, AAV10, AAV11, or AAV-2i8 VP1, VP2 and/or VP3 capsid proteins, or comprises a modified or variant VP1, VP2 and/or VP3 capsid protein selected from any of: AAV1, AAV2, AAV3, AAV4, AAV5, AAV6, AAV10, AAV11, and AAV-2i8 AAV serotypes.

67. (canceled)

68. A composition according to claim 3, wherein the cells are subcultured to a cell density in the range of about 0.1×10⁶ cells/ml to about 5.0×10⁶ cells/ml prior to contact with said plasmid/PEI mixture.

69. A composition according to claim 68, wherein the cells are contacted with said plasmid/PEI mixture between a period of 2 days to 5 days after subculture.

70. A composition according to claim 68, wherein the cells are contacted with said plasmid/PEI mixture between a period of 3 days to 4 days after subculture.

71. (canceled)

72. A method according to claim 22, wherein the amount of recombinant AAV vector produced is at least 50% or greater with the step of adding Free PEI to the plasmid/PEI cell culture compared to without adding Free PEI to the plasmid/PEI cell culture.

73. (canceled)


SEQUENCE LISTING (3 sequences)

SEQ ID NO: 1 - DNA, 21 bp, Artificial Sequence
  Description of Artificial Sequence Forward primer
gcacaagctggagtacaacta

SEQ ID NO: 2 - DNA, 19 bp, Artificial Sequence
  Description of Artificial Sequence Reverse primer
tgttgtggcggatcttgaa

SEQ ID NO: 3 - DNA, 24 bp, Artificial Sequence
  Description of Artificial Sequence Probe
agcagaagaacggcatcaaggtga